(19) United States
(12) Patent Application Publication
(10) Pub. No.: US 2017/0068794 A1
(43) Pub. Date: Mar. 9, 2017

(54) DEVICES, SYSTEMS AND METHODS FOR DIAGNOSTIC TESTING

(71) Applicant: 3PDx, LLC, Salt Lake City, UT (US)

(72) Inventors: William Robert Pagels, Salt Lake City, UT (US); Andrew David Pagels, Salt Lake City, UT (US); Christopher William Pagels, Salt Lake City, UT (US)

(21) Appl. No.: 15/093,579

(22) Filed: Apr. 7, 2016

Related U.S. Application Data
(63) Continuation of application No. 14/412,417, filed on Dec. 31, 2014, now abandoned.
(60) Provisional application No. 61/666,992, filed on Jul. 2, 2012.
(60) Provisional application No. 61/666,997, filed on Jul. 2, 2012.
(60) Provisional application No. 61/667,002, filed on Jul. 2, 2012.
(60) Provisional application No. 61/706,686, filed on Sep. 27, 2012.
(60) Provisional application No. 61/721,998, filed on Nov. 2, 2012.
(60) Provisional application No. 61/724,063, filed on Nov. 8, 2012.

(51) Int. Cl. G06F 19/00; G01N 33/569

(52) U.S. Cl. CPC G06F 19/3418; G01N 33/56988; G01N 33/56911; G01N 2469/10

(57) ABSTRACT

The present disclosure relates to devices, systems, and methods for performing diagnostic tests. The disclosed diagnostic devices are capable of performing analytic tests and communicating with a portable multifunctional device (PMD) or other computing device. Through input and manipulation of materials within the diagnostic device, a large range of tests may be performed. For example, alteration or customization of chemical components of the diagnostic device may enable many analytic applications to be provided. These analytic tests may include, but are not limited to, sensing or quantification of chemicals from sample input, whether gaseous, liquid, or otherwise, sensing or quantification of analytes, antibodies, or antigens, sensing or quantification of genetic material, or other substances.

### CROSS-REFERENCE TO RELATED APPLICATIONS

This patent application claims priority to U.S. Provisional Patent Application No. 61/667,002 entitled CONNECTION BETWEEN PORTABLE MULTIFUNCTIONAL DEVICE AND AN EXTERNAL DEVICE, filed on Jul. 2, 2012, U.S. Provisional Patent Application No. 61/666,992 entitled METHOD AND DEVICE FOR PERFORMING DIAGNOSTIC TESTS, filed on Jul. 2, 2012, U.S. Provisional Patent Application No. 61/666,997 entitled METHOD FOR USER CONTROL OF DIAGNOSTIC TESTS, filed on Jul. 2, 2012, U.S. Provisional Patent Application No. 61/706,686 entitled DIAGNOSTIC TEST SYSTEMS, DEVICES and RELATED COMPONENTS AND METHODS, filed on Sep. 27, 2012, U.S. Provisional Patent Application No. 61/721,998 entitled SYSTEMS AND METHODS OF PRESENTING SUPPORT RESOURCES, filed on Nov. 2, 2012, and U.S. Provisional Patent Application No. 61/724,063 entitled SYSTEMS AND METHODS FOR DIAGNOSTIC TESTING, filed on Nov. 8, 2012, each of which is incorporated herein by reference in its entirety.

### TECHNICAL FIELD

The present disclosure is directed to devices, systems, and methods for diagnostic testing involving a computing device. More specifically, the disclosure is directed towards devices, systems, and methods for performing analytic tests with a diagnostic device that is configured to communicate with a portable multifunctional device (PMD) or other computing device.

### BRIEF DESCRIPTION OF THE DRAWINGS

The embodiments disclosed herein will become more fully apparent from the following description and appended claims, taken in conjunction with the accompanying drawings. While various aspects of the embodiments are presented in drawings, the drawings depict only typical embodiments, which will be described with additional specificity and detail through use of the accompanying drawings in which:

FIG. 1 depicts a system for diagnostic testing, according to one embodiment of the present disclosure.

FIG. 2 depicts a diagnostic device, according to another embodiment of the present disclosure.

FIGS. 3A and 3B depict an illustrative representation of electrochemical detection, according to another embodiment of the present disclosure.

FIG. 4 depicts a diagnostic device, according to another embodiment of the present disclosure.

FIGS. 5A-5C depict a diagnostic device, according to another embodiment of the present disclosure.

FIGS. 6A-6C depict a system for diagnostic testing, according to another embodiment of the present disclosure.

FIGS. 7A-7C depict various sample carriers that may be used with a diagnostic device, according to another embodiment of the present disclosure.

FIG. 8 depicts a sample carrier and a diagnostic device, according to another embodiment of the present disclosure.

FIG. 9 depicts a sample carrier and a diagnostic device, according to another embodiment of the present disclosure.

FIGS. 10A-10C depict various views of a sample carrier and a diagnostic device, according to another embodiment of the present disclosure.

FIGS. 11A and 11B depict a sample carrier and a diagnostic device, according to another embodiment of the present disclosure.

FIGS. 12A-12C depict a sample carrier, according to another embodiment of the present disclosure.

FIGS. 13A-13C depict a sample carrier, according to another embodiment of the present disclosure.

FIGS. 14A-14C depict a sample carrier, according to another embodiment of the present disclosure.

FIGS. 15A-15C depict a system for diagnostic testing, according to another embodiment of the present disclosure.

FIG. 16 depicts a system for diagnostic testing, according to another embodiment of the present disclosure.

FIGS. 17A-17C depict various systems for diagnostic testing, according to other embodiments of the present disclosure.

FIG. 18 depicts a flow diagram of a method for diagnostic testing, according to another embodiment of the present disclosure.

FIG. 19 depicts a flow diagram of a method for diagnostic testing, according to another embodiment of the present disclosure.

FIG. 20 depicts a flow diagram of a method for preparing for diagnostic testing, according to another embodiment of the present disclosure.

FIG. 21 depicts a flow diagram of a method for diagnostic testing, according to another embodiment of the present disclosure.

FIG. 22 depicts a flow diagram of a method for accessing a support network based on diagnostic testing results, according to another embodiment of the present disclosure.

FIG. 23 depicts an electrical signal generated from an exemplary diagnostic test, according to another embodiment of the present disclosure.

FIG. 24 depicts an enlarged portion of the electrical signal of FIG. 23.

FIG. 25 depicts an electrical signal generated from an exemplary diagnostic test, according to another embodiment of the present disclosure.

FIG. 26 depicts an electrical signal generated from an exemplary diagnostic test, according to another embodiment of the present disclosure.

FIG. 27 depicts an enlarged portion of the electrical signal of FIG. 26.

FIGS. 28A-28B depict an illustrative representation of electrochemical detection, according to another embodiment of the present disclosure.

FIGS. 29A-29B depict an illustrative representation of electrochemical detection, according to another embodiment of the present disclosure.

### DETAILED DESCRIPTION OF PREFERRED EMBODIMENTS

Developments in diagnostics, smart phones, and wireless communication are converging on a new way of conducting diagnostics. Just one example of the huge role smart phones and disseminated diagnostics technology will play in our lives in the future is the multitude of medical applications that have been created to serve the growing population of smart phone users. Of the almost one million medical apps available, over 80% are geared towards exercise and biometrics. The majority of the remaining percentage comprises reference applications that are static and cannot freely accept, interpret, or give out personalized information about the user. Additionally, most patients diagnosed for a particular medical issue do not immediately have access to a tailored treatment program or to a support system surrounding that treatment. As more than 50% of Americans own a smart phone, with that number expected to exceed 60% by the end of 2014, quality healthcare in the form of powerful, simple, affordable tools on handheld or other portable computing devices may usher in a new paradigm of connection between individuals that harnesses the potential of the present digital revolution.

The present disclosure relates to devices, systems, and methods for performing diagnostic tests. As set forth in detail below, the disclosed diagnostic devices are capable of performing analytic tests and communicating with a portable multifunctional device (PMD) or other computing device. For example, in some embodiments, the coupling and/or connection between a diagnostic device and a PMD allows a user to access and utilize a multitude of rapid, user-friendly, and portable testing platforms. Further, a wide range of settings and/or testing parameters may be employed and the need for conventional analytic and diagnostic hardware and/or equipment may be minimized or negated, resulting in reduced medical costs and increased portability and accessibility of diagnostic tests.

In some embodiments, the diagnostic device comprises one or more components or elements, which may also be described as componentry. Through utilization of the components, one or more diagnostic assays or tests may be conducted. The components may include chambers or wells, channels, gates, pumps, electrical systems, electrodes, sensors, etc., and/or combinations thereof. Some components may be configured to move materials including reagents and/or samples (e.g., control samples and/or test samples) through the diagnostic device. Other components may be configured to monitor and/or measure signals (e.g., electrical signals). Other components may be configured to transfer or move signals throughout the diagnostic device.

In some embodiments, the diagnostic device may be configured to function and/or operate independently, without the need for additional external testing equipment. For example, a signal (e.g., an electrical signal) may be produced within the diagnostic device that is proportional to the present of an analyte in a sample of interest, the rate of formation of a species, and/or a combination thereof. This signal may be transmitted to and processed by a PMD and may be used to produce a test result.

In some embodiments, the systems disclosed herein may comprise an interface for user control. For example, an interface may comprise software or other graphical user interface contained within a PMD. In another embodiment, firmware or hardware, either separate from or as part of the PMD, may be utilized to allow user control of the diagnostic device.

In some embodiments, users of tools implemented on a PMD or other computing device may perform many functions, due to multiple capabilities incorporated in these tools. The PMD may be coupled to an external diagnostic device. The tools may facilitate interaction with the diagnostic device, such as collection of test results from the diagnostic device.

One function of the disclosed tools may allow a user to interact with the PMD to control an external device connected to the PMD. The external device may be a diagnostic device that functions by receiving electrical stimulus from the PMD. The electrical stimulus may power mechanical and chemical processes on the diagnostic device.

Another function of the tools may be to facilitate measurement and reception of data signals from the diagnostic device before, during, and after operation of the diagnostic device. For example, the PMD may receive data from the diagnostic device in the form of, for example, electrical signals. The data may come in the form of a rate of change in electrical signal, or may be gleaned by absolute measurement at different points throughout testing. The data may be interpreted by the PMD to represent distinct, objective test results, and these results may be displayed on the PMD, for example, for a user to view.

The disclosed tools may have capability of packaging and transmitting test results gained from the coupling of the PMD and the diagnostic device. The packaged test results may be transmitted to, for example, entities outside the PMD environment, such as statistics and/or tracking organizations, service providers, including physicians, and the like. The packaged test results data may be redacted to remove identifying information about the user. The packaged test results data may also include identifying information about the user. These test results may constitute a basis for use of information housed within the software, or connection to third parties. In other words, information may be presented to the user based on the test results.

The disclosed tools may present to a user a listing of support resources relevant to the test results, including, for example, service professionals and equipment and other suppliers. The user may be able to navigate the presented resources, searching based upon several criteria. For example, a user may filter the presented resources by proximity, and may specify an annular area (e.g., zip code and/or area code) around them (e.g., a present location, a home location, work location, etc.) from which to return resources (e.g., an area greater than a certain first distance away from a given location and within a further second distance away from the user). Users may also filter resources by quality ratings, as generated by other users.

Additional aspects and advantages will be apparent from the following detailed description of preferred embodiments, which proceeds with reference to the accompanying drawings. Embodiments disclosed in the figures are generally presented in terms of a medical diagnostic, but it will be appreciated that the disclosed embodiments may be applicable to a variety of testing applications, such as for example regulatory compliance (radioactivity levels, emission/pollution levels, HAZMAT), security (TSA explosive testing, bio surveillance, bio testing), law enforcement (breath/blood alcohol level/concentration, substance identification), etc. The scope of the present disclosure is not limited to medical diagnostic testing.

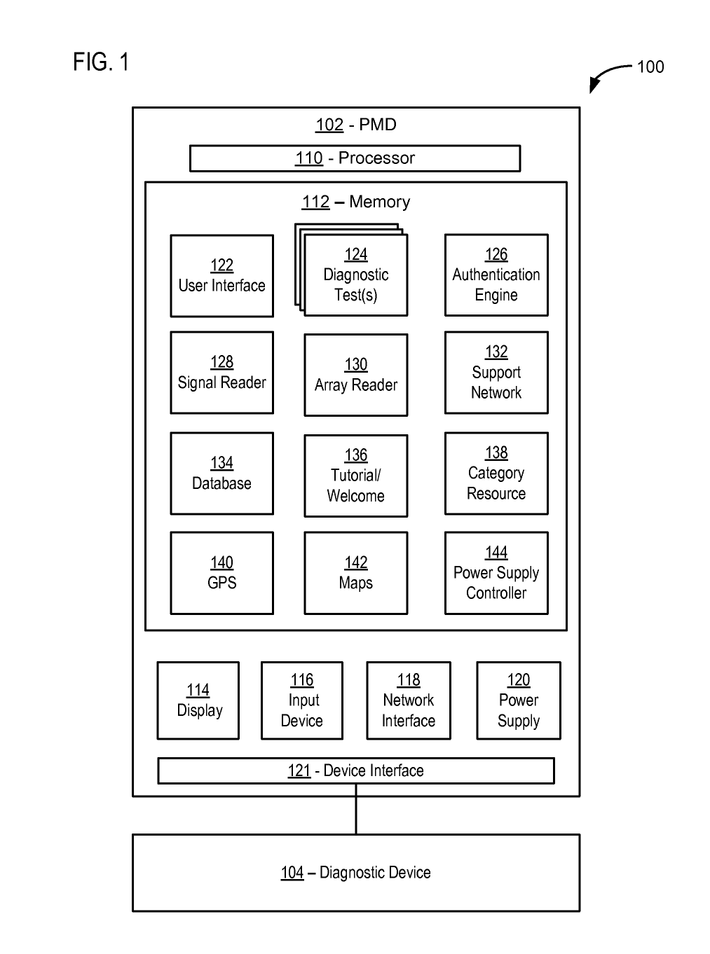

FIG. 1 depicts a system 100 for diagnostic testing, according to one embodiment of the present disclosure. The system 100 may include a computing device, such as a PMD 102, and a diagnostic device 104 configured to couple to the computing device. Through input and manipulation of materials within the diagnostic device 104, a large range of tests may be performed by the system 100. For example, alteration or customization of chemical components of the diagnostic device 104 may enable many analytic applications to be provided. These analytic tests may include, but are not limited to, sensing or quantification of chemicals from sample input, whether gaseous, liquid, or otherwise, sensing or quantification of analytes, antibodies, or antigens, sensing or quantification of genetic material, or other substances.

A user interface 122 may be included on the PMD 102 that may allow the user to control some aspects of the diagnostic device 104, and may present the results or measurements obtained from the diagnostic device 104 to the user. This user interface 122 may also provide information about resources, organizations, or people to the user, which may be of interest, assistance, or support to the user in reference to and/or based on a diagnostic test result.

The PMD 102 may include, but is not limited to, an iPhone, an Android telephone, or another “smart” mobile telephone; an iPad, an Android tablet, or other tablet device; a computer, PDA, or portable computer (e.g. laptop), or another PMD or “smart” mobile device. In other embodiments, the PMD may be a desktop computing device. In still other embodiments, the PMD may be a customized and/or specific computing device.

The PMD 102 may provide a plurality of functions related to the diagnostic device 104. The PMD 102 may control or enable operation of the diagnostic device 104, either through automated phone control, manual control from the user through the PMD 102, or a combination of both. The PMD 102 may provide power to the diagnostic device 104, which may actuate the diagnostic device 104, and in some instances, allow for movement of components or materials within in the diagnostic device 104. For example, in some embodiments, the PMD 102 may 1) power and/or control fluid pump and valve systems on the diagnostic device 104 that may be used to control the movement of reagents, solutions, suspensions and/or other liquids on the diagnostic device 104; 2) power circuitry and/or electrical systems on the diagnostic device 104; 3) power a mechanism to transfer a sample such as a fluid from a sample carrier such as an absorbent swab, in some instances by squeezing; 4) provide power to resistors to create temperature changes (such as may be required for thermal cycling); 5) provide power to mix and/or rehydrate components necessary to interact to produce a measurable signal; 6) supply electricity for electrochemical detection; 7) supply power to purify suspensions through an on-device filtration process and so forth. In some embodiments, for example, electrical current may be supplied to the diagnostic device 104 from the PMD 102 through one or more connection points. Similarly, function commands and other inputs may be received by the diagnostic device 104 through electrical or other connections with the PMD 102.

The PMD 102 may also control functions on a self-powered diagnostic device 104 or device that derives power from an external source other than the PMD 102. The PMD 102 may house and run a software interface, which may allow the user to control aspects of the diagnostic device 104, view test results, access information about resources in reference to these test results, and communicate test results and associated user information to other data collection sites or to service providers. The PMD 102 may receive electronic signals from the diagnostic device 104 related to the materials within the diagnostic device 104 and process these signals, and may display this processed data to the user through, for example, a user interface 122.

The PMD 102 may include a processor 110, a memory 112, a display 114, an input device 116 (e.g., a keypad, microphone, etc.), a network interface 118, a power supply 120 (e.g., a battery), and a device interface 121 (e.g., a docking port or other communication coupling mechanism).

The PMD 102 may further include a plurality of modules or other components configured to perform a variety of functions and/or operations for diagnostic testing. The modules may be stored in the memory 112, as shown in FIG. 1. In other embodiments, the modules may comprise hardware components.

The modules or components may include, but are not limited to, a user interface 122, one or more test modules 124, an authentication engine 126, a signal reader 128, an array reader 130, a support network module 132, a database 134, a tutorial/welcome module 136, a category resource engine 138, a global positioning system (GPS) component 140, a maps engine 142, a power supply controller 144, and other components.

The user interface 122 may present information on the display 114 and facilitate user input via the input device 116.

The one or more test modules 124 may be embodied as a test engine. The one or more test modules 124 may generate and display (e.g., via the user interface 122 on the display 114) instructions on procedures associated with performing a diagnostic test through a plurality of mechanisms, and may trigger other modules or components.

The authentication engine 126 may read unique signatures from the diagnostic device 104 inserted into the PMD, and may generate and display forms in which the user may add input, or which may be static forms. The authentication engine 126 may also trigger other modules or components.

The signal reader 128 may read, process, or interpret electronic signals at pins of the device interface 121 (or port) of the PMD 102 that may correspond to diagnostic information. The signal reader 128 may also trigger other modules or components.

The array reader 130 may read, process, or interpret information or data contained within arrays of data generated by other modules or components. The array reader 130 may also trigger other modules or components.

The support network module 132 may trigger and control various other modules or components that may allow the user to identify, locate, and access data describing resources contained within the support network module 132 and/or or third parties. The support network module 132 may also trigger other modules or components.

The database 134 may store data and/or forms in which the user may add input, or which may be static forms. The database 134 may also read, process, interpret, package, and transmit user input into arrays stored within application or memory 112 or may transmit to third parties via the internet. The database 134 may also trigger other modules or components.

The tutorial or welcome module 136 may generate and display forms in which the user may add input, or which may be static forms. The tutorial or welcome module 136 may also retrieve data and display data, including, but not limited to, text, images, and videos that may instruct use of (or interaction with) other modules or components. The tutorial or welcome module 136 may also trigger other modules or components.

The category resource engine 138 may generate and display forms in which the user may add input, or which may be static forms. The category resource engine 138 may also retrieve data and display data including but not limited to text, images, and videos. The category resource engine 138 may generate and display location-specific information based upon other hardware and/or software in the PMD 102 (e.g., such as a GPS component 140). The category resource engine 138 may also trigger other modules or components.

The GPS component 140 enables capture, acquisition, and/or generation of location information.

The maps engine 142 may manage and present maps, for example, in connection with displaying location information generated by the GPS and/or location information of resources as specified in, for example, the database.

The power supply controller 144 may operate to determine and/or provide power from the power supply 120 to the diagnostic device 104.

The diagnostic device 104 may provide a plurality of functions to the user. The diagnostic device 104 may be configured to receive a sample and run a diagnostic test on the sample. The diagnostic device 104 may further be configured to run a control and/or calibration test. The diagnostic device 104 may receive an electronic signal from electrodes or other sensor and transmit this signal to the PMD 102. Furthermore, the diagnostic device 104 may be powered by current or a battery or fuel cell, may receive power from the PMD 102, or other means to provide the energy necessary to move components and/or materials within the diagnostic device 104. The diagnostic device 104 may further comprise an interface and/or connection system that is compatible with the PMD 102.

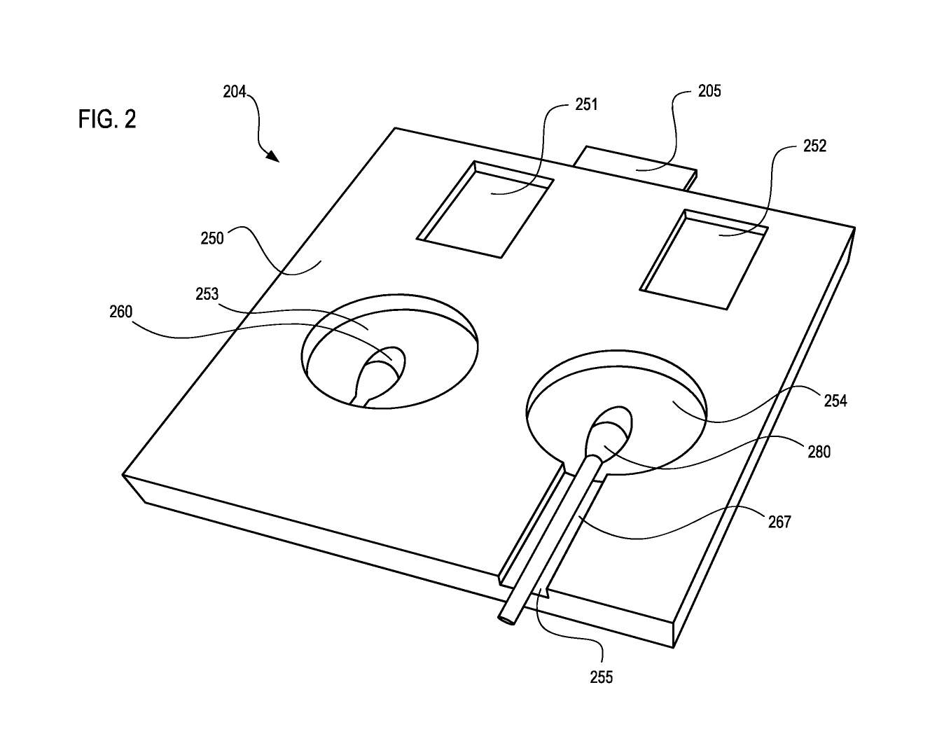

FIG. 2 depicts a diagnostic device 204, according to another embodiment of the present disclosure. As shown in FIG. 2, the diagnostic device 204 may comprise a connector 205 that is configured to mate with or otherwise couple to a PMD or other computing device. For example, the connector 205 may be configured to mate with a computer bus, input/output port, power port, and/or other communication port of a PMD. In some embodiments, the connector 205 may be compatible with an input/output port on a smart phone (e.g., iPhone, Android telephone, etc.) or other smart mobile device (iPad, tablet etc.). In some embodiments, the connector 205 may be configured to couple with an Apple Lightning connection interface. In some embodiments, the connector 205 may be configured to couple with a 30-pin connection interface. In yet other embodiments, the connector 205 may be configured to couple with a standard or miniature universal serial bus (USB) connection interface. Electrical power, electrical signals (e.g., input/output signals), and so forth may pass between the diagnostic device 204 and the PMD via the connector 205.

As further shown in FIG. 2, the diagnostic device 204 comprises a housing 250, which may be referred to as a body member or casing structure. The housing 250 may be composed of various materials. For example, the housing may comprise polymeric materials (e.g., plastics), metallic materials, glass materials, and/or combinations thereof. Other materials may also be used.

The housing 250 may be configured to retain one or more components, including chambers or wells 251, 252, 253, 254, channels, gates, valves, pumps (e.g., polymer pumps, user-operated pumps, etc.), cranks, buttons, electrodes, sensors, and/or electrical systems. The components may be configured for use in performing a test, such as a diagnostic test, chemical, or compound detection test, etc. The components may be mechanical, electromechanical, electrical, magnetic, electromagnetic, chemical, fluorescent, colorimetric, and/or electrochemical in nature. The components may also be various sizes, including micro-level components and nano-level components. In some embodiments, one or more components may be molded into or otherwise integrally formed with the housing 250.

The components may be configured to serve a variety of functions. In some embodiments, one or more components may be configured for use in the insertion of a test sample into the diagnostic device 204. Additionally, one or more components may be configured for use in the manipulation (e.g., movement, mixing, etc.) of a test sample and/or control sample, or other material within the diagnostic device. One or more components may be configured to allow passage of test samples and/or control samples through portions of the diagnostic device. One or more components may be configured to monitor, detect, and/or measure electrical signals. One or more components may be configured to transfer electrical signals throughout the diagnostic device. In some embodiments, one or more components may also be configured to house or retain other components.

With continued reference to FIG. 2, in the illustrated embodiment, the housing 250 comprises four chambers 251, 252, 253, 254. The chambers 251, 252, 253, 254 may be integrally formed within the housing 250 and may have various conformations. The chambers 251, 252, 253, 254 may also serve a variety of purposes. For example, the housing 250 may comprise one or more reagent chambers 251, 252, and/or one or more sample chambers 253, 254. In the illustrated embodiment, the housing 250 comprises first and second reagent chambers 251, 252. The reagent chambers 251, 252 may be configured to house, store, or otherwise retain chemical reagents. The chemical reagents may comprise liquid reagents, and may include reactants, reaction products, and/or buffer solutions.

In the illustrated embodiment, the housing 250 further comprises first and second sample chambers 253, 254. More specifically, the housing 250 comprises a test sample chamber 254, and a control sample chamber 253. The sample chambers 253, 254 may be configured to receive a sample, including a test sample and/or a control sample. In some embodiments, the sample chambers 253, 254 may be configured to house and/or retain the sample during a diagnostic test. In some embodiments, the sample chambers 253, 254 may be configured to house and/or retain an electrode or other sensor through which an electrical current may be monitored, detected and/or measured during a diagnostic test. In some embodiments, the diagnostic testing substantially occurs within the sample chambers 253, 254.

In some embodiments, the diagnostic device 204 may comprise one or more components which may be stimulated to manipulate the materials within the diagnostic device 204. For example, one or more components may be stimulated to regulate movement of a reagent from a reagent chamber 251, 252 to a sample chamber 253, 254. The components may be electrically, chemically, and/or mechanically stimulated. For example, in some embodiments, the diagnostic device 204 comprises polymers and/or gels comprising electroactive polymers (EAPs), ionic polymer metal composites (IPMCs), and/or other electronically-activated materials that may expand and/or contract in response to electronic stimuli that may come from a PMD. In some embodiments, expansion and/or contraction of the electronically-activated polymers and/or gels may be utilized to transfer a reagent from a reagent chamber 251, 252 to a sample chamber 253, 254. In some embodiments, the expansion and/or contraction of the electronically-activated polymers and/or gels may be utilized to mix or otherwise manipulate the materials within the diagnostic device 204, for example, to initiate a diagnostic testing sequence. In some embodiments, the electronically-activated polymers and/or gels may be referred to as polymer pumps.

The diagnostic device 204 may comprise one or more manually stimulated and/or user-operated components, including pumps, cranks, and/or buttons. The user-operated components may be utilized to transfer a reagent from a reagent chamber 251, 252 to a sample chamber 253, 254. The user-operated components may further be utilized to mix or otherwise manipulate materials within the diagnostic device, for example, to initiate a diagnostic testing sequence. Through user-operated components, a user may generate the forces needed for input of materials into the device and also manipulate materials within the device. In some embodiments, a combination of one or more polymer pumps and one or more user-operated components may be used.

The polymer pumps and/or user-operated components may be disposed in a reagent chamber 251, 252, sample chamber 253, 254 and/or other areas within the diagnostic device 204. For example, in some embodiments, a reagent chamber 251, 252 may be configured to house or retain a polymer pump that is configured to push, move or otherwise force a reagent from the reagent chamber 251, 252 and into a sample chamber 253, 254. The reagent, once pushed into the sample chamber 253, 254, may initiate a biochemical process resulting in the analysis of a sample. In some embodiments, the sample may be disposed on a test sample carrier 280 and/or a control sample carrier 260. In some embodiments, the sample carrier 260, 280 may comprise an absorbent swab.

In some embodiments, one or more polymer pumps and/or user-operated components may be configured to manipulate and/or move a reagent comprising an buffer solution, which may also be referred to as an eluent buffer solution, eluent buffer, eluent solution, solvent solution, etc. For example, polymer pumps and/or user-operated components may force a buffer solution through one or multiple channels or passageways within the diagnostic device 204. For example, a buffer solution may be moved from a reagent chamber 251, 252 to a sample chamber 253, 254 via one or more channels. In some embodiments, the buffer solution may be configured to elute a sample contained within and/or on a sample carrier 280. For example, a buffer solution may be used to elute a sample contained within an absorbent swab of a sample carrier 280. Once eluted, the sample may interact with one or more components contained within the sample chamber 253, 254, including sensors, capture probes and/or electrodes during a diagnostic analysis. The buffer solution may also be used to rehydrate a sample within the sample chamber 253, 254. In some embodiments, the sample chamber 253, 254 may also contain electronically-activated polymers or other gels, which may be stimulated to expand and/or contract in order to mix the contents of the sample chamber 253, 254, which may include the buffer solution and the sample. The sample chamber 253, 254 may further contain and/or be surrounded by other components which may be configured to alter the conditions of the chamber 253, 254, such as but not limited to temperature, pressure, or other conditions.

As shown in FIG. 2, the diagnostic device 204 further comprises one or more sample introduction ports 255 which may be disposed within the housing 250. A sample introduction port 255 may be configured to receive a test sample such as a gaseous, liquid, fluid, or solid test sample. In some embodiments, the sample introduction port 255 is configured to receive a test sample that is disposed on a sample carrier 280. For example, the sample introduction port 255 may be configured to receive an absorbent swab. The sample introduction port 255 may also be configured to receive a test tube or other sample container. In some embodiments, the sample introduction port 255 may be in communication with a test sample chamber 254. Once the test sample is positioned within the test sample chamber 254, a diagnostic test sequence may be initiated, which may include movement of a reagent from a reagent chamber 251, 252 into the sample chamber 253, 254 via, for example, one or more polymer pumps.

In some embodiments, the sample introduction port 255 may be configured to be sealed after insertion of the sample carrier 280. For example, the sample introduction port 255 may comprise a pliable and/or deformable seal through which a sample carrier 280 may be inserted. In some embodiments, a lid that may be made of the same material as the diagnostic device 204 (or any another material) may close to form a seal around the sample carrier 280. Other methods of sealing the sample carrier 280 within the sample diagnostic device 204 may also be used. Once sealed, the sample carrier 280 may be held in place by the seal and/or the channel or passageway 267, which leads to a test sample chamber 254. The sample carrier 280 may provide a means of transporting a plurality of samples through this channel or passageway, to undergo a plurality of diagnostic tests once inserted.

In some embodiments, the diagnostic device 200 may be configured to be a consumable device. For example, the diagnostic device 200 may be a single use device, or a device configured for limited use (e.g., 2 uses, 3 uses, 4 uses, 5 uses, etc.) In other embodiments, the diagnostic device 200 is configured as a non-consumable device that can be reused as desired.

Various detection methods may be employed by the diagnostic device 204. In some embodiments, electrochemical detection methods may be employed. In some embodiments, for example, the diagnostic device 204 comprises a reagent that may be disposed within the reagent chambers 251, 252. The reagent may comprise redox conjugate compounds. Other compounds that may bind chemical entities of interest in the sample may also be used. Illustrative redox conjugate compounds may include conjugates such as ferrocene, an HRP/H₂O₂/hydroquinone system, or another redox system or organometallic compound. The reagent may further comprise a chemical buffer in which samples, redox conjugate compounds, and/or other reagent compounds may dissolve.

Upon initiation of a diagnostic test sequence, the reagent may be transferred from the reagent chamber 251, 252 to the sample chambers 253, 254 for example, via one or more polymer pumps. The sample chambers 253, 254 may comprise a system by which presence of a chemical entity creates an increase or decrease in electrical signal, which may be accomplished by the presence of a chemical capture probes bound to one or more electrodes or sensors. The capture probes may bind the reagent (e.g., a reagent comprising a redox conjugate compound), the chemical entity of interest, or a combination thereof, which may cause either an increase in the transfer of electrons to the electrode or sensor, a decrease in the transfer of electrons to the electrode or sensor, or a combination thereof. This “molecular wire” system may include, but is not limited to, the chemical entity of interest, the reagent (e.g., a reagent comprising redox conjugate compounds), capture probes, or other compounds known to one of ordinary skill in the art.

The increase or decrease in electrical signal may be transferred down through this “molecular wire” to the electrodes or sensors, which may pass the electronic signal to a PMD either wirelessly or through one or more wires. The signal may be quantified through measurement by a voltmeter, an ammeter, or another electronic measurement device located on the diagnostic device 204 or elsewhere, and may be processed by the PMD, and then may be displayed to the user, optionally in a plurality of formats, to provide information about the contents of the test sample.

In some embodiments, the electrode or other sensor may be bound and/or coupled to capture probes, which may comprise a peptide and/or another chemical entity. The chemical entity may allow indirect and/or direct binding of the peptide to the electrode. For example, the chemical entity may comprise a thiolated hydrocarbon chain, which may be bound to the N-terminus of a peptide. The C-terminus of the peptide may be modified and bound with a plurality of chemical agents, including but not limited to a redox agent such as methylene blue. In some embodiments, the peptide may have a chemical affinity for one or multiple entities in the sample solution. When there is no bond between these entities and the peptide, the peptide may be highly flexible, and may efficiently achieve electron transfer to and from the redox agent. When there is a bond between these entities and the peptide, the peptide may become less flexible, and, in binding this entity, may lose the ability or efficiency of electron transfer to and from the redox agent through a plurality of mechanisms, including, but not limited to, being physically and chemically obstructed by the bound entity, or moved a sufficient distance away from electrode. In some embodiments, the diagnostic device 204 also comprises a solution that is capable of unbinding the peptide from the entity.

In other embodiments, the electrode may comprise a DNA sensor such as, in some embodiments, an aptamer. In such embodiments, the electrical conductivity of DNA and/or other oligonucleotide constructs is dependent on its conformational state. For example, upon binding or otherwise incorporating an analyte from a sample, the conformation of the DNA sensor may switch, thereby resulting in an altered conductive path between two oligonucleotide stems. An electrode or other sensor may be used to monitor the electron transfer. This methodology electrochemical detection is further described in U.S. Pat. Nos. 7,947,443 and 7,943,301, each of which is incorporated by reference.

In other embodiments, the detection method may comprise colorimetry and/or fluorimetry. For example, the diagnostic device 200 may comprise a colorimeter and/or a fluorometer. The colorimeter and/or fluorometer may be coupled to other components within the diagnostic device 200, and may be used to analyze various sample types.

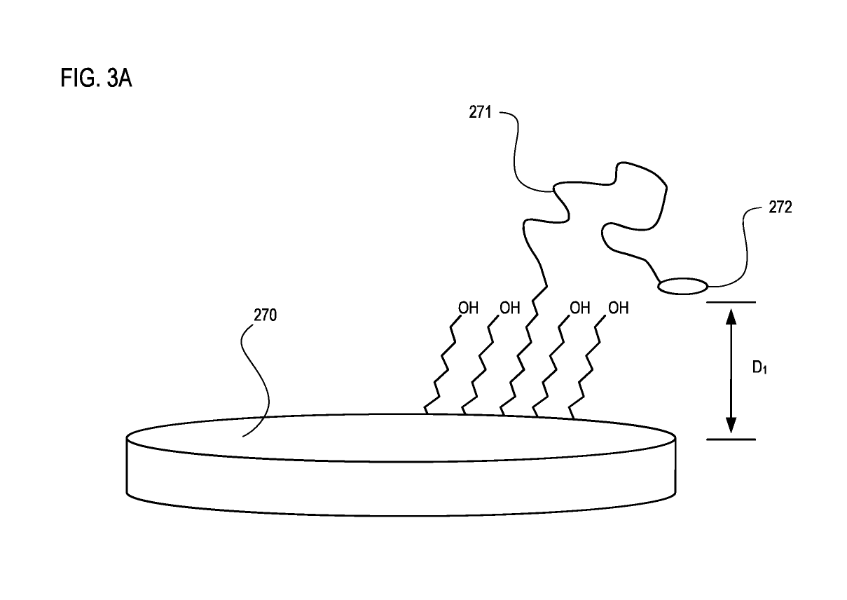

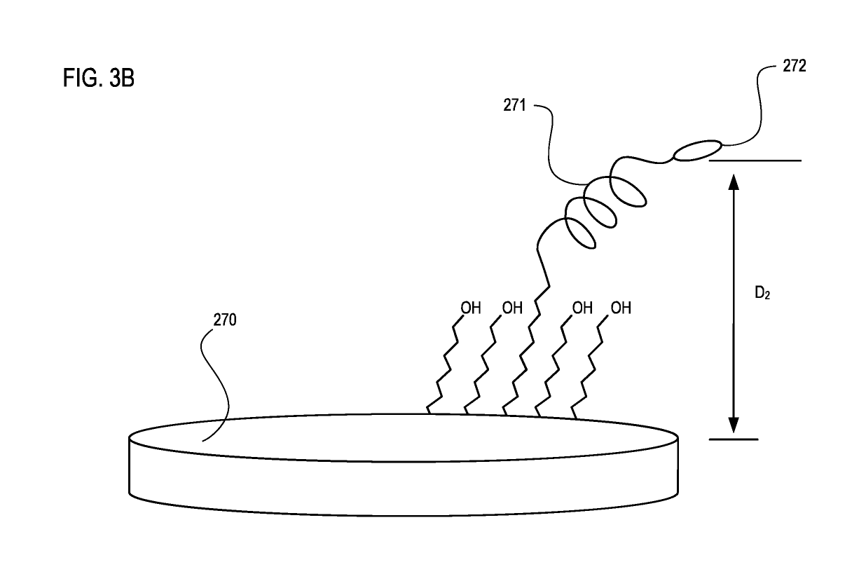

FIGS. 3A and 3B depict an illustrative representation of electrochemical detection, according to another embodiment of the present disclosure. In particular, FIGS. 3A and 3B depict an electrode 270 that may be configured to measure the transfer of electrons during a diagnostic test. Referring both to the diagnostic device 204 shown in FIG. 2 and to structural diagrams shown in FIGS. 3A and 3B, in some embodiments, the diagnostic device 204 may be sensitized to a specific diagnostic species as a consequence of the biochemical components immobilized or contained with the sample chambers 253, 254. For example, for a HIV test, HIV-specific peptides or proteins are immobilized to an electrode 270 that is disposed in the bottom of sample chambers 253, 254. In one embodiment, the HIV-specific peptide or protein 271 changes conformation upon binding a HIV antibody in the patient or control sample that is introduced via the sample carrier 260, 280 from an amorphous structure to a polypeptide chain with defined structure (such as a alpha helix or beta strand or beta sheet). Bound to this peptide is a redox-sensitive moiety 272 that when attached to the amorphous peptide, demonstrates a very high electron transfer rate (high k_{ET}) in communication with the PMD. Upon antibody binding, the redox-sensitive moiety moves away from the electrode and the k_{ET} is dramatically reduced. For example, as shown in FIGS. 3A and 3B, distance D₂ is greater than distance D₁. As a consequence of the change in k_{ET} as detected by the PMD, this mechanism can be utilized for quantifying antibodies in a patient sample.

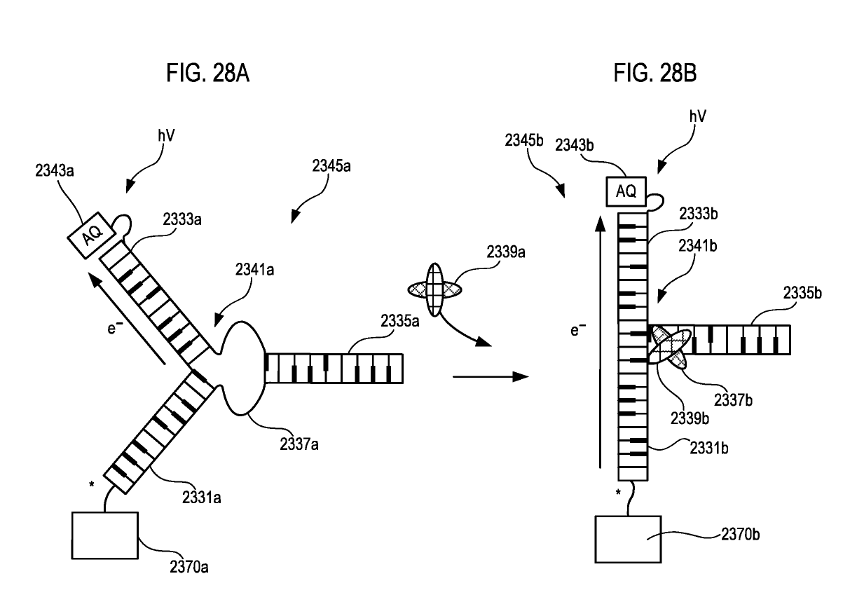

FIGS. 28A and 28B depict an illustrative representation of electrochemical detection, according to another embodiment of the present disclosure. In particular, FIG. 28A depicts the sensor system 2345a in an unbound state (first conformational state), and FIG. 28B depicts the sensor system 2345b in a bound state (second conformational state). As shown in FIGS. 28A and 28B, a first oligonucleotide stem 2331a, 2331b and a second oligonucleotide stem 2333a, 2333b are connected together at a junction 2341a, 2341b. Stems 2331a, 2331b, 2333a, 2333b may comprise double helical DNA, or other nucleic acid constructs. The sensor system 2345a, 2345b may further comprise a third oligonucleotide stem 2335a, 2335b. The sensor system 2345a, 2345b further comprises a receptor 2337a, 2337b, which may form part of the junction 2341a, 2341b. The receptor 2337a, 2337b may comprise a nucleic acid aptamer sequence selected to bind to a target analyte.

In the illustrated embodiment, first stem 2331a, 2331b functions as an electron donor and second stem 2333a, 2333b functions as an electron sink (although the reverse configuration may also be employed). When an analyte 2339a, 2339b binds to a receptor 2337a, 2337b, a conformation change in the sensor system 2345a, 2345b occurs, resulting in a detectable change in charge transfer between the first and second stems 2331a, 2331b, 2333a, 2333b. The conformational change may consist of adaptive folding, compaction, structural stabilization or some other steric modification of junction in response to analyte 2339a, 2339b binding which causes a change in the charge transfer characteristics of the sensor system 2345a, 2345b.

As further illustrated in FIGS. 28A and 28B, in some embodiments, the sensor system 2345a, 2345b may comprise a charge flow inducer 2343a, 2343b, which may comprise antraquinone (AQ) or rhodium (III) complexes with aromatic ligands, for controllably inducing charge transfer between first and second stems 2331a, 2331b, 2333a, 2333b in the second conformational state. Additionally, the sensor system 2345a, 2345b may be coupled to or otherwise attached to an electrode 2370a, 2370b that is disposed within a sample chamber of the diagnostic device. This methodology electrochemical detection is further described in U.S. Pat. Nos. 7,947,443 and 7,943,301, each of which is incorporated by reference.

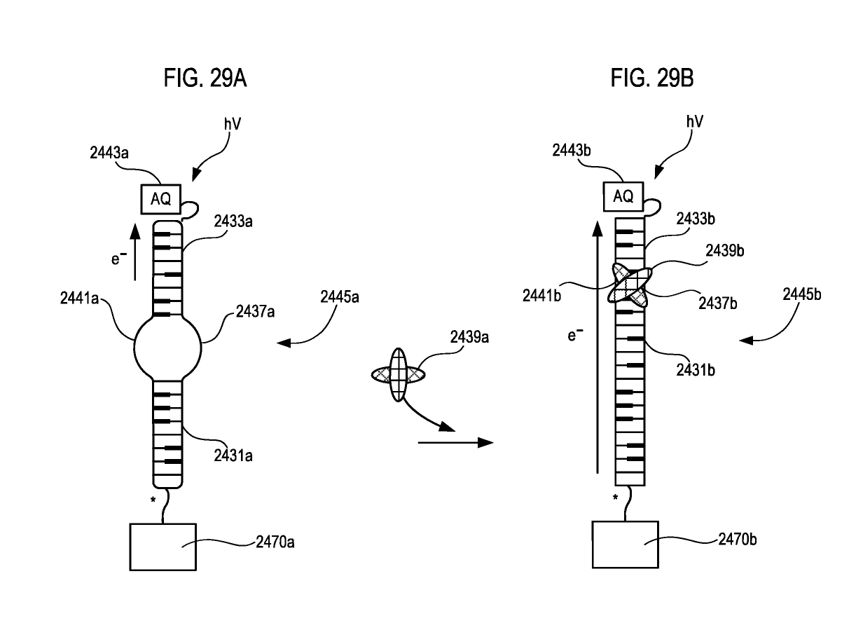

FIGS. 29A and 29B depict an illustrative representation of electrochemical detection, according to another embodiment of the present disclosure. In particular, FIG. 29A depicts the sensor system 2445a in an unbound state (first conformational state), and FIG. 29B depicts the sensor system 2445b in a bound state (second conformational state). As shown in FIGS. 29A and 29B, a first oligonucleotide stem 2431a, 2431b and a second oligonucleotide stem 2433a, 2433b are connected together at a junction 2441a, 2441b. The sensor system 2445a, 2445b further comprises a receptor 2437a, 2437b, which may form part of the junction 2441a, 2441b.

In the illustrated embodiment, first stem 2431a, 2431b functions as an electron donor and second stem 2433a, 2433b functions as an electron sink (although the reverse configuration may also be employed). When an analyte 2439a, 2439b binds to a receptor 2437a, 2437b, a conformation change in the sensor system 2445a, 2445b occurs, resulting in a detectable change in charge transfer between the first and second stems 2431a, 2431b, 2433a, 2433b. For example, prior to the binding of the analyte 2439a, 2439b, charge transfer between first and second stems 2431a, 2431b, 2433a, 2433b may be substantially impeded.

As further illustrated in FIGS. 29A and 29B, in some embodiments, the sensor system 2445a, 2445b may comprise a charge flow inducer 2443a, 2443b for controllably inducing charge transfer between first and second stems 2431a, 2431b, 2433a, 2433b in the second conformational state. Additionally, the sensor system 2445a, 2445b may be coupled to or otherwise attached to an electrode 2470a, 2470b that is disposed within a sample chamber of the diagnostic device. This methodology electrochemical detection is further described in U.S. Pat. Nos. 7,947,443 and 7,943,301, each of which is incorporated by reference.

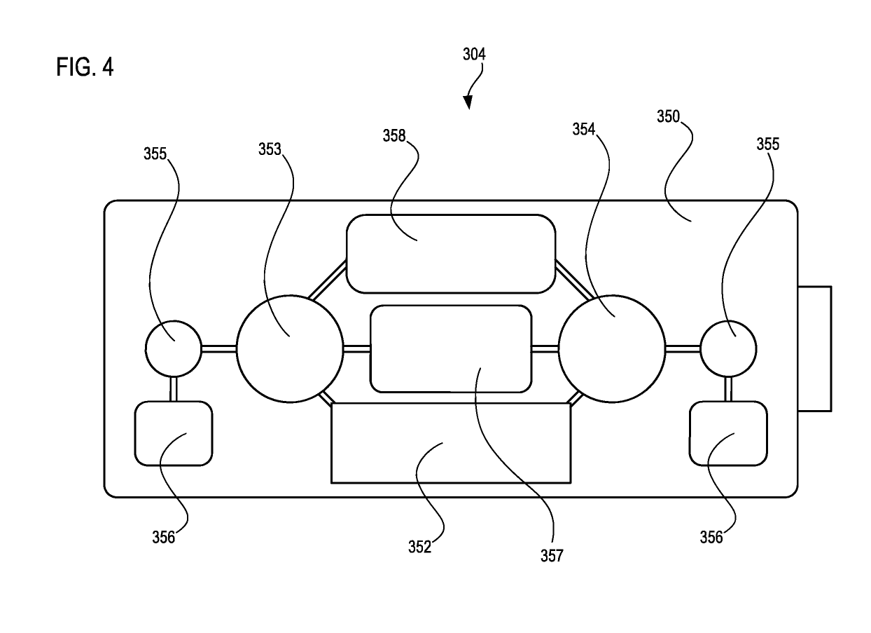

FIG. 4 depicts a diagnostic device 304 according to another embodiment of the present disclosure. As shown in FIG. 4, the diagnostic device 304 may comprise a housing 350 that that comprises first and second sample chambers 353, 354. In some embodiments, the diagnostic test sequence may comprise a multi-step ELISA type reaction in which a series of biochemical and wash reagents are added to sample chambers 353, 354. Test samples and/or control samples may be introduced to the diagnostic device 304 via sample introduction ports 355, 356. The test samples and/or control samples may thereafter be introduced into the sample chambers 353, 354, for example, via a reagent comprising a reaction buffer contained within one or more buffer chambers 356. A biochemical reagent, such as an unlabeled peptide (271 of FIGS. 3A and 3B) or DNA sensor may be contained with a reagent chamber 351, 352 and pumped into the sample chambers 353, 354 at the appropriate step in the analytical reaction sequence. This step may be followed by a subsequent step of adding an electrochemical modulator such as ferrocene from yet another chamber, 357. In some embodiments, waste may be collected in a waste chamber 358.

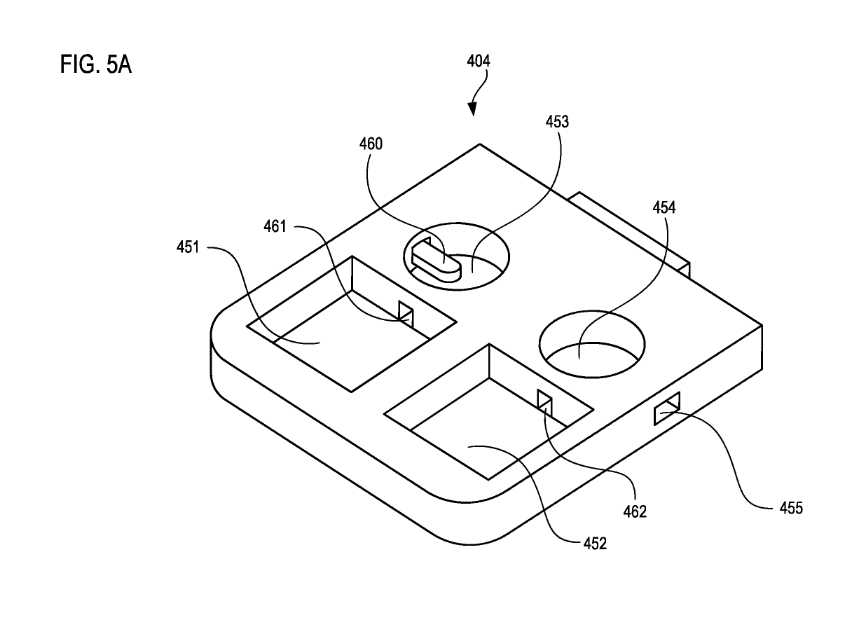

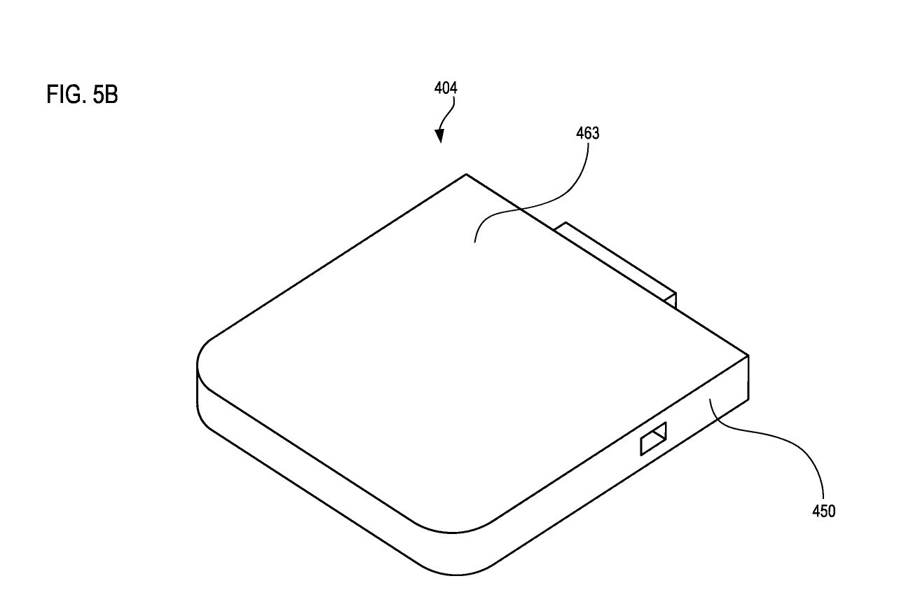

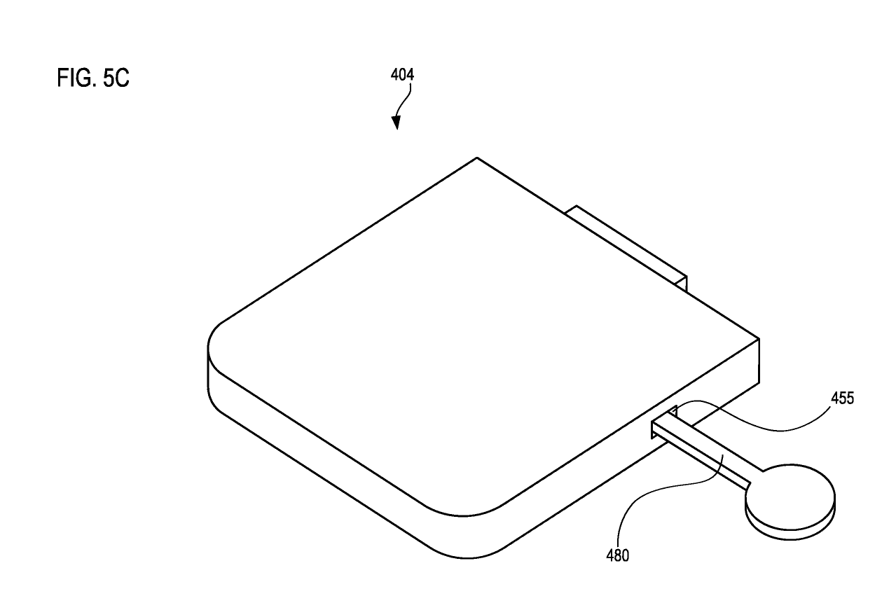

FIGS. 5A, 5B, and 5C depict a diagnostic device 404 according to another embodiment of the present disclosure. As shown in FIG. 5A, the diagnostic device 404 may comprise a plurality of reagent chambers 451, 452. In some embodiments, the reagent chambers 451, 452 may be configured to accommodate polymer pumps. In the illustrated embodiment, the reagent chambers 451, 452 are in fluid communication with the sample chambers 453, 454. In particular, a first reagent chamber 451 is in fluid communication with the control sample chamber 453 via a channel or passageway 461. A second reagent chamber 452 is in fluid communication with the test sample chamber 454 via a second channel or passageway 462. The channels 461, 462 may allow for passage of reagents including elution buffers and/or other solutions between the reagent chambers 451, 452 and the sample chambers 453, 454.

The diagnostic device 404 further comprises a control sample carrier 460. The control sample carrier 460 may comprise an absorbent swab comprising a control sample. As shown in the illustrated embodiment, the control sample carrier 460 may be coupled, affixed, embedded, or otherwise attached to a sidewall of the control sample chamber 453.

The diagnostic device 404 further comprises a sample introduction port 455 that is in fluid communication with the test sample chamber 454. The sample introduction port 455 may be configured to receive a test sample carrier, and may allow for insertion of the test sample carrier into the test sample chamber 454.

FIG. 5B depicts the diagnostic device 404 of FIG. 5A according to another embodiment of the present disclosure. As shown in FIG. 5B, the housing 450 may comprise a solid lid 463 or other type of covering. FIG. 5C depicts the diagnostic device of FIGS. 5A and 5B wherein a test sample carrier 480 has been inserted into the sample introduction port 455.

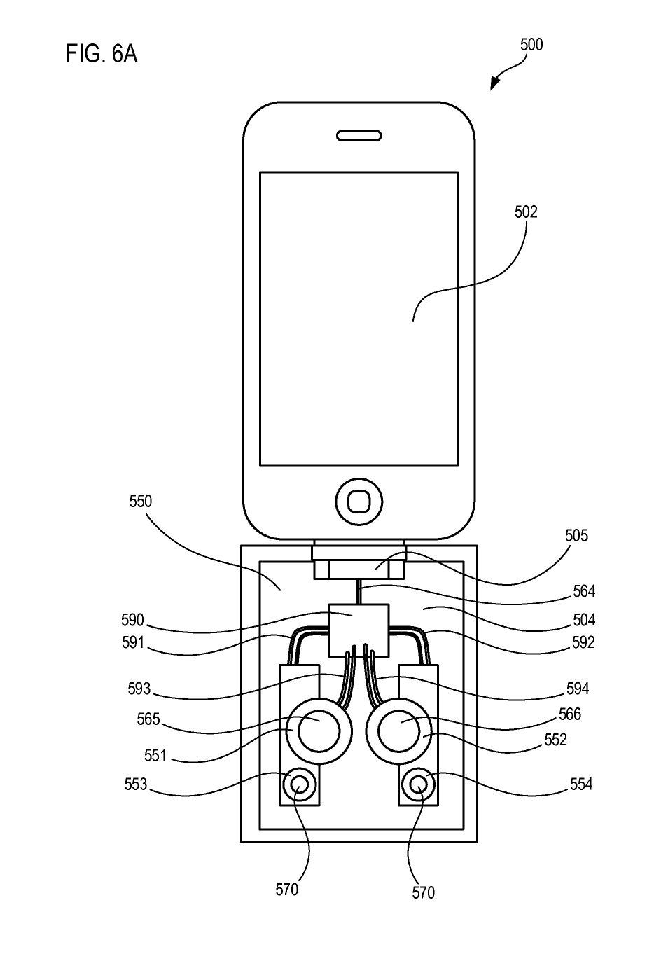

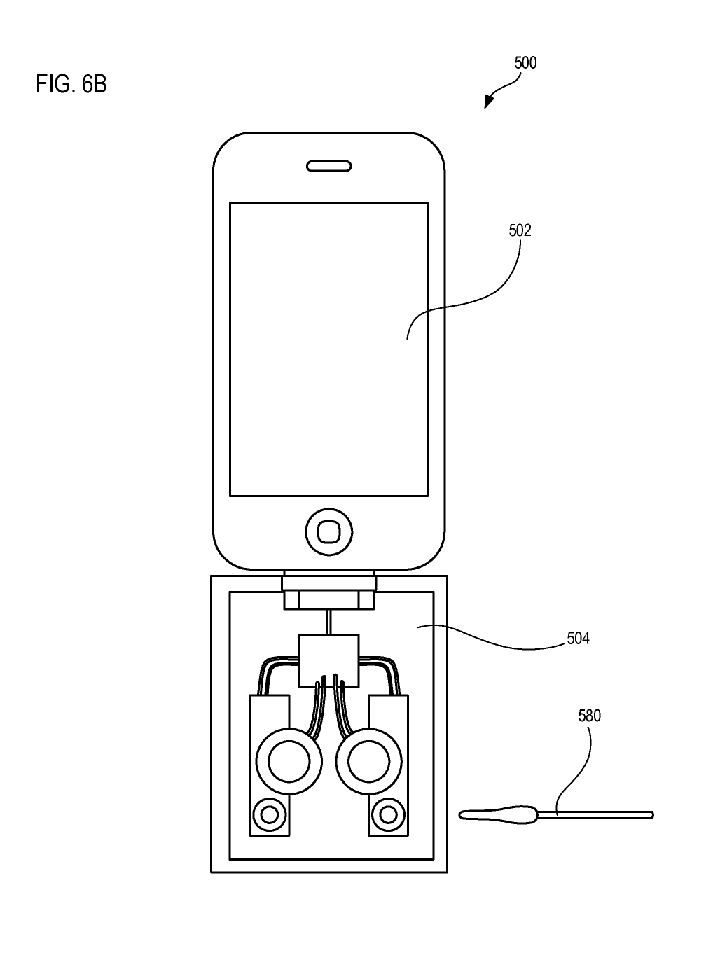

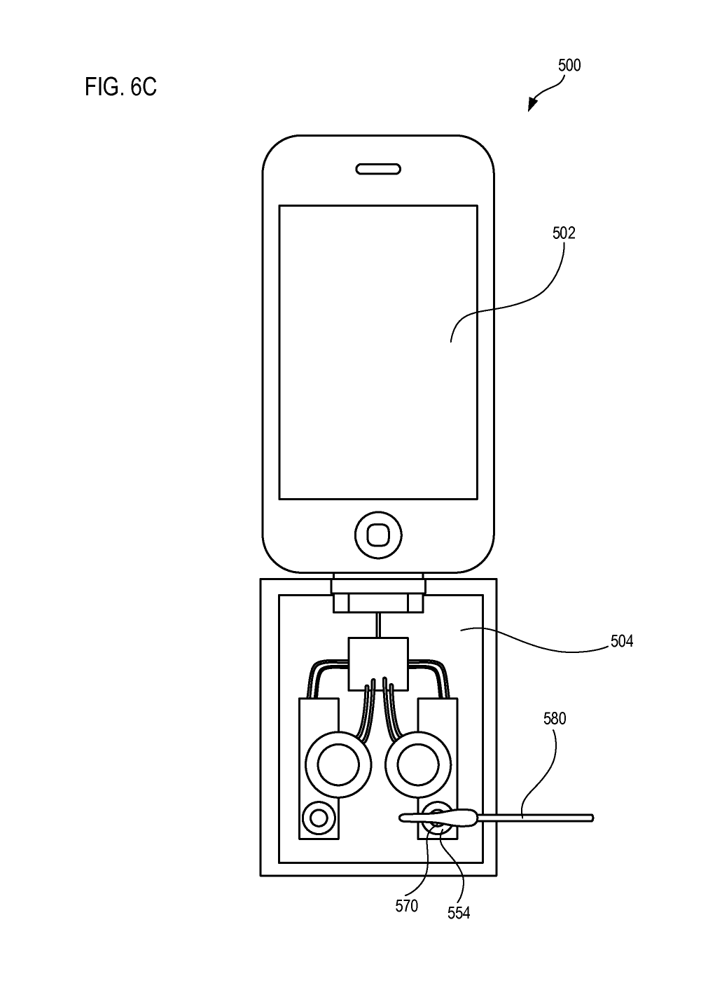

FIGS. 6A, 6B, and 6C depict a system 500 for diagnostic testing, according to another embodiment of the present disclosure. In the illustrated embodiment, the system 500 comprises a PMD 502 coupled to a diagnostic device 504. The diagnostic device 504 may be coupled to the PMD 502 in various ways. As shown in the illustrated embodiment, the diagnostic device 504 may be coupled to the PMD 502 via a wired or physical connection, which may be referred to as a physical connecting mechanism. In such embodiments, the PMD 502 may comprise a coupling interface. In some embodiments, the coupling interface may comprise a computer bus, input/output port, power port, and/or other communication port. The diagnostic device 504 may comprise a complimentary connector 505 that is configured to mate with the coupling interface of the PMD 502. For example, in the illustrated embodiment, the PMD 502 is coupled to the diagnostic device 504 via a connector 505 that is configured to mate with the PMD 502. The connector 505 may comprise one or more wires and/or other communication protocols that are necessary for the facilitation and transfer of electrical signals (e.g., digital data and/or power) between the PMD 502 and the diagnostic device 504. Various types of connectors 505 may be used.

The wired connection may be established by direct connection of the diagnostic device 504 to the PMD 502 via one or more wires, wire cables, and/or other physical devices (e.g., connectors, etc.). In some embodiments, the diagnostic device 504 is coupled to a docking port and/or other communication port of the PMD 502. In other embodiments, the PMD 502 and the diagnostic device 504 may be coupled via one or more intermediate devices.

In other embodiments, the diagnostic device 504 may be coupled to the PMD 502 via a wireless connection, which may be referred to as a virtual connection mechanism. For example, the diagnostic device 504 may be coupled to the PMD 502 through one or more wireless networks and/or through local signaling from the PMD 502 to the diagnostic device 504. The diagnostic device 104 may be coupled to the PMD 502 via Bluetooth or other wireless communication protocols, or via any area network, whether local or otherwise.

In the illustrated embodiment, the diagnostic device 505 comprises a housing 550 and an electrical system 590 disposed within the housing 550. The diagnostic device 505 further comprises first and second reagent chambers 551, 552, a control sample chamber 553, and a test sample chamber 554, each of which is disposed within the housing 550. The diagnostic device 505 further comprises a first polymer pump 565 and a second polymer pump 566 disposed within the first and second reagent chambers 551, 552 respectively. In some embodiments, the polymer pumps 565, 566 may be electrically actuated and/or activated, via, for example, an electrical signal originating from the PMD 502. The diagnostic device 505 further comprises electrodes 570 disposed within the sample chambers 553, 554.

The electrical system 590 may comprise hardware, software, standard electrical components and/or circuitry, and one or more processors and/or microprocessors. The electrical system 590 may be electrically coupled to the connector 505 via one or more wires 564 or other electrical pathway. The electrical system 590 may also be electrically coupled to the polymer pumps 565, 566 and electrodes 570. For example, the electrical system 590 may be electrically coupled to the polymer pumps 565, 566 via one or more wires 593, 594 or other electrical pathways. Similarly, the electrical system 590 may be electrically coupled to the electrodes 570 via one or more wires 591, 592 or other electrical pathways.

In some embodiments, the electrical system 590 may be configured to send electrical signals to and/or receive electrical signals from the PMD 502. The electrical system 590 may further be configured to send electrical signals to and/or receive electrical signals from various components within the diagnostic device 504. For example, the electrical system 590 may send electrical signals to one or more polymer pumps 551, 552 which may actuate the polymer pumps 551, 552. The electrical system 590 may also receive electrical signals from the electrodes 570 or other sensors. In some embodiments, the electrical system 590 may further be configured to process electrical signals either from the PMD 502, or from one or more components within the diagnostic device 504.

FIG. 6B depicts the system 500 of FIG. 6A comprising a PMD 502 coupled to a diagnostic device 504. Additionally, FIG. 6B illustrates a sample carrier 580 prior to being introduced into the diagnostic device 504. FIG. 6C depicts the system 500 of FIG. 6A after a sample carrier 580 has been introduced into the diagnostic device 504. As shown in FIG. 6A, the sample carrier 580 has been inserted into the diagnostic device 504 such that it is partially disposed over the sample test chamber 554 and an electrode 570. Following insertion of the sample carrier 580, a diagnostic test may be initiated, for example, via a signal from the PMD 502.

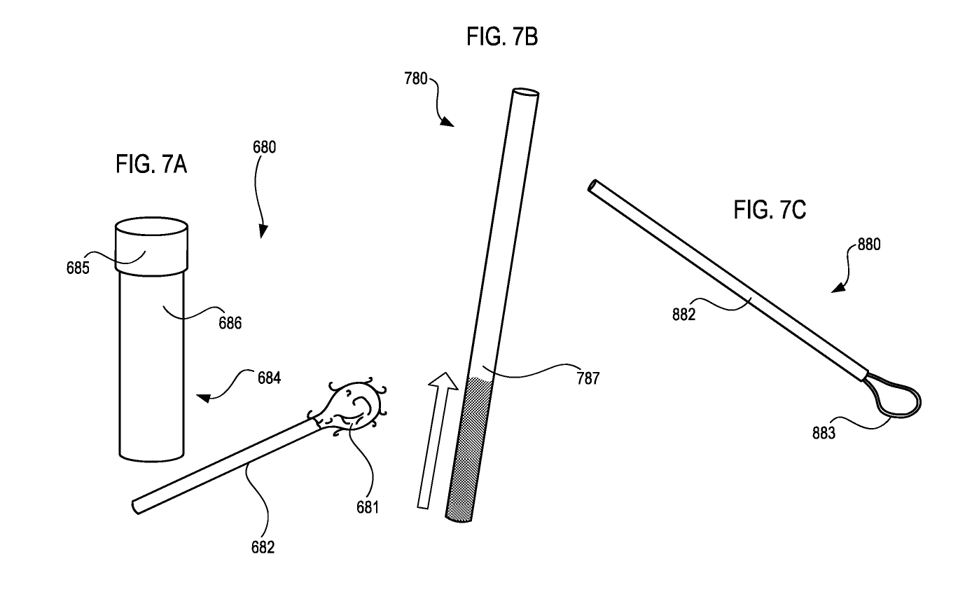

FIGS. 7A, 7B, and 7C depict several sample carriers 680, 780, 880 that may be used according to various embodiments of the present disclosure. For example, in FIG. 7A, the sample carrier 680 comprises an absorbent swab 681 coupled to a handle, stick, or shaft 682. In some embodiments, the absorbent swab 681 may be a flocked swab comprising nylon. Other absorbent materials may be used. The absorbent swab 618 may be configured to absorb a sample prior to delivery to a diagnostic device. The absorbent swab 681 may thereafter be inserted into a diagnostic device. In some embodiments, a buffer solution contained within the diagnostic device may be used to elute the sample from the absorbent swab 681 following insertion of the sample carrier 680 into the diagnostic device. In other embodiments, one or more components of the diagnostic device may be configured to squeeze and/or otherwise release the sample from the absorbent swab 681 and into a test sample chamber of the diagnostic device.

In some embodiments, the sample carrier 680 may be disposed within a container 648. The container 648 may be at least partially filled with a buffer solution or other solvent. As shown in the illustrated embodiment, the container 684 may comprise a test tube 686 and a cap 685. The cap 685 may be configured to seal or close the test tube 686 either reversibly, or irreversibly. In some embodiments, the cap 685 may be screwed or twisted onto the test tube 686. In other embodiments, the cap 685 may be snapped onto the test tube 685 via a snap fit connection.

The buffer solution within the container 648 may be configured to elute the sample out of the sample carrier 680. For example, the buffer solution within the container 648 may elute the sample out of the absorbent swab 681 following insertion of the sample carrier 680 into the container 648. The elution may occur prior to and/or during a diagnostic test.

In some embodiments, the container 684 may be configured for use without a separate sample carrier. For example, a solid sample may be disposed and dissolved in the buffer solution within the container 684. The container 684 may thereafter be introduced to a diagnostic device and an analysis of the sample may be performed.

FIG. 7B depicts a sample carrier 780 according to another embodiment of the present disclosure. As shown in FIG. 7B, the sample carrier 780 may comprise a capillary tube 787. As indicated by the reference arrow, a fluid sample may be drawn into the capillary tube 787 and collected via capillary action. A solid sample may also be collected in the capillary tube 787, if desired. In some embodiments, the capillary tube 787 may be disposed into a container comprising a buffer solution (such as the container 684 depicted in FIG. 7A) prior to being delivered to a diagnostic device. In other embodiments, the capillary tube 787 may be delivered directly to a diagnostic device for diagnostic testing.

FIG. 7C depicts yet another embodiment of a sample carrier 880 according to the present disclosure. As shown in FIG. 7C, in some embodiments, the sample carrier comprises a handle 882 and a terminating loop 883. The loop 883 may collect a plurality of samples (e.g., fluid and/or solid samples). In some embodiments, the loop 883 may be disposed into a container comprising a buffer solution (such as the container 684 depicted in FIG. 7A) prior to being delivered to a diagnostic device. In other embodiments, the sample carrier 880 comprising the loop 883 may be delivered directly to a diagnostic device for diagnostic testing.

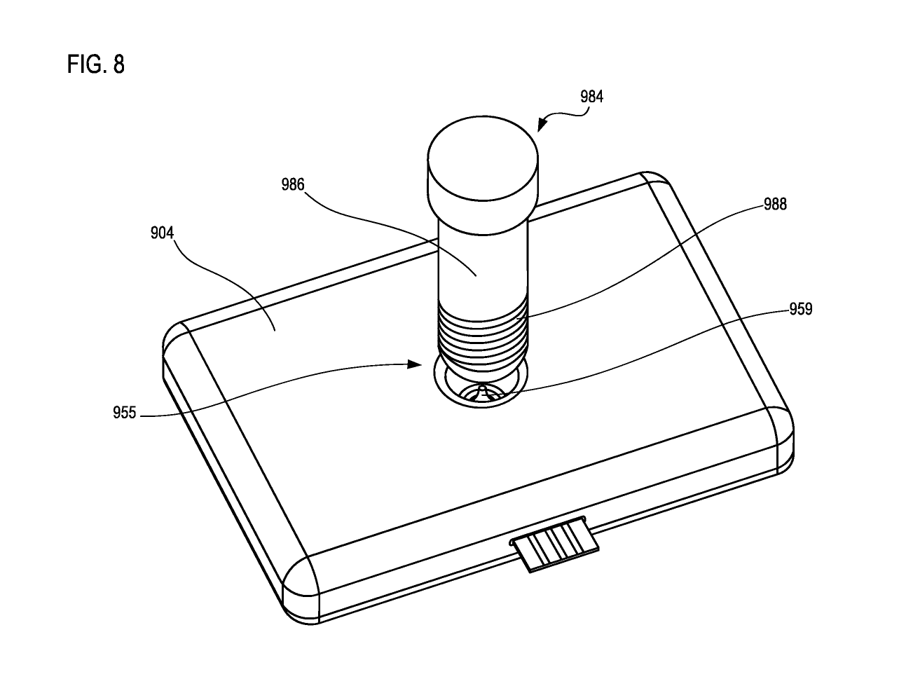

FIG. 8 depicts a diagnostic device 904 according to another embodiment of the present disclosure. In the illustrated embodiment, the diagnostic device 904 is configured to receive a container 984 within which a test sample may be disposed or dissolved. As previously discussed, in some embodiments, a sample carrier may be disposed within the container 984; and in other embodiments, the container 984 may independently function as the sample carrier.

As shown in FIG. 8, in some embodiments, the container 984 may comprise a test tube 986 that comprises one or more threads 988. The threads 988 may be disposed along a portion of the outer periphery of the test tube 986. The threads 988 may be configured to engage with complimentary threads disposed around the sample introduction port 955. In such embodiments, the container 984 may be coupled to the diagnostic device 904 via a threaded engagement.

The diagnostic device 904 may further comprise a puncturing device 959. The puncturing device 959 may be disposed near the sample introduction port 955, and may be configured to puncture or rupture a wall of the container 984. After a wall of the container 984 is ruptured, the buffer solution from within the container 984 (including a test sample) may be dispensed out of the container 984 and into the diagnostic device. For example, the buffer solution comprising a test sample may be dispensed directly, or through one or more channels, into a test sample chamber wherein a diagnostic test may be performed. In the illustrated embodiment, the puncturing device 959 is configured to rupture a bottom wall of the container 984 as the container 984 is twisted (i.e., threaded) inwardly through the sample introduction port 955.

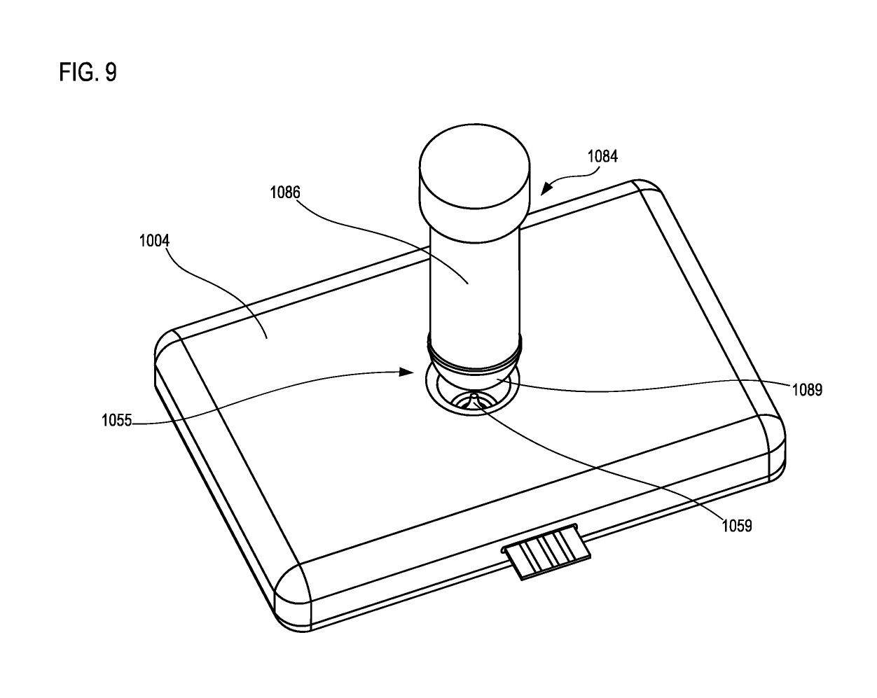

FIG. 9 depicts a diagnostic device 1004 according to another embodiment of the present disclosure. In the illustrated embodiment, the diagnostic device 1004 is configured to receive a container 1084 within which a test sample may be disposed or dissolved. As previously discussed, in some embodiments, a sample carrier may be disposed within the container 1084; and in other embodiments, the container 1084 may independently function as the sample carrier.

As shown in FIG. 9, in some embodiments, the container 1084 may comprise a test tube 1086. In some embodiments, a fitting or ring 1089 may be disposed around the outer periphery of a portion of the test tube 1086. The test tube 1086, or the fitting 1089 on the test tube 1086, may be configured to engage with the outer walls of the sample introduction port 1055. In such embodiments, the container 1084 may be coupled to the diagnostic device 1004 via this engagement such that the container 1084 is not easily removable. In other embodiments, the sample introduction port 1055 may comprise a polymeric ring 1089 or other component that may be configured to retain a container 1084 that has been inserted into the diagnostic device 1004.

The diagnostic device 1004 may further comprise a puncturing device 1059. The puncturing device 1059 may be disposed near the sample introduction port 1055, and may be configured to puncture or rupture a wall of the container 1084. After a wall of the container 1084 is ruptured, the buffer solution from within the container 1084 (including a test sample) may be dispensed out of the container 1084 and into the diagnostic device. For example, the buffer solution comprising a test sample may be dispensed directly, or through one or more channels, into a test sample chamber wherein a diagnostic test may be performed. In the illustrated embodiment, the puncturing device 1059 is configured to rupture a bottom wall of the container 1084 as the container 1084 is slid or otherwise inserted inwardly through the sample introduction port 1055.

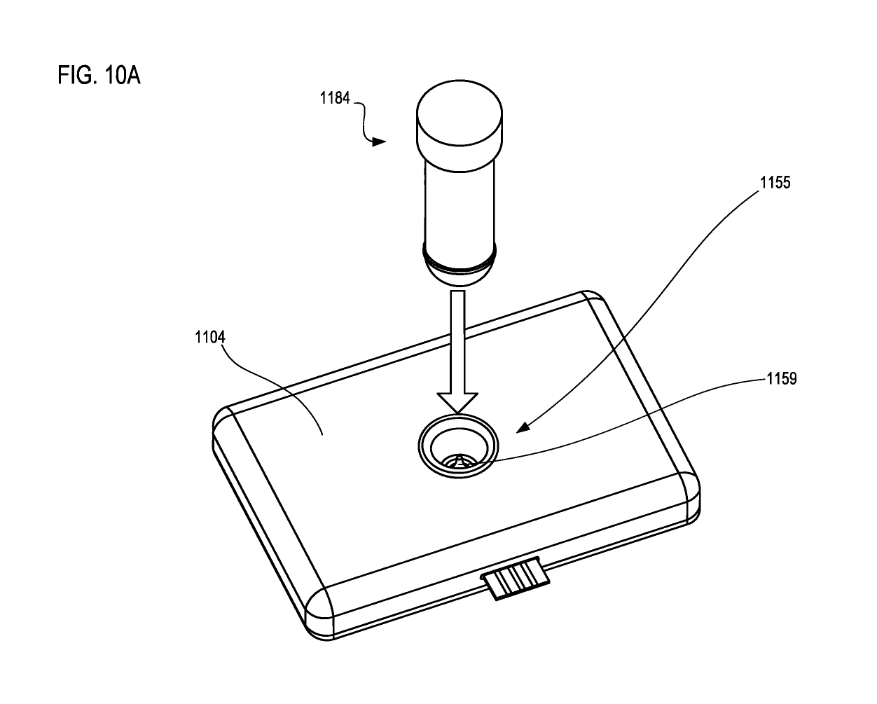

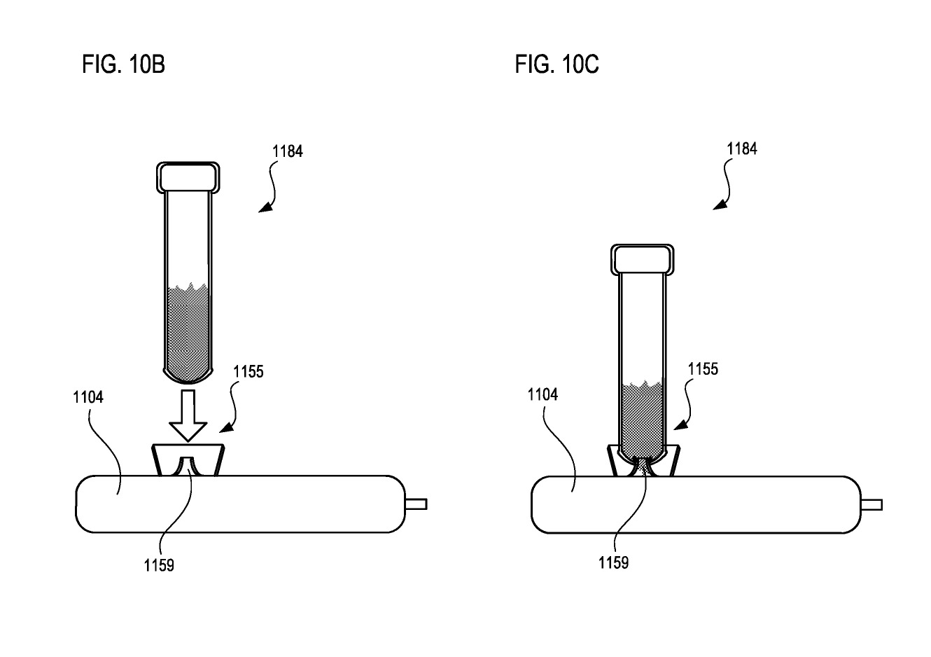

FIGS. 10A-10C depict a sample carrier 1180 being inserted into a diagnostic device 1104 according to another embodiment of the present disclosure. As shown in FIGS. 10A-10C, a container 1184 containing a test sample may be slid or otherwise inserted inwardly through a sample introduction port 1155, as indicated by the reference arrow of FIGS. 10A and 10B. FIG. 10C illustrates the puncturing device 1159 rupturing through the bottom wall of the container 1184, which may cause the contents of the container 1184, including a buffer solution and test sample, to be dispensed into the diagnostic device 1104.

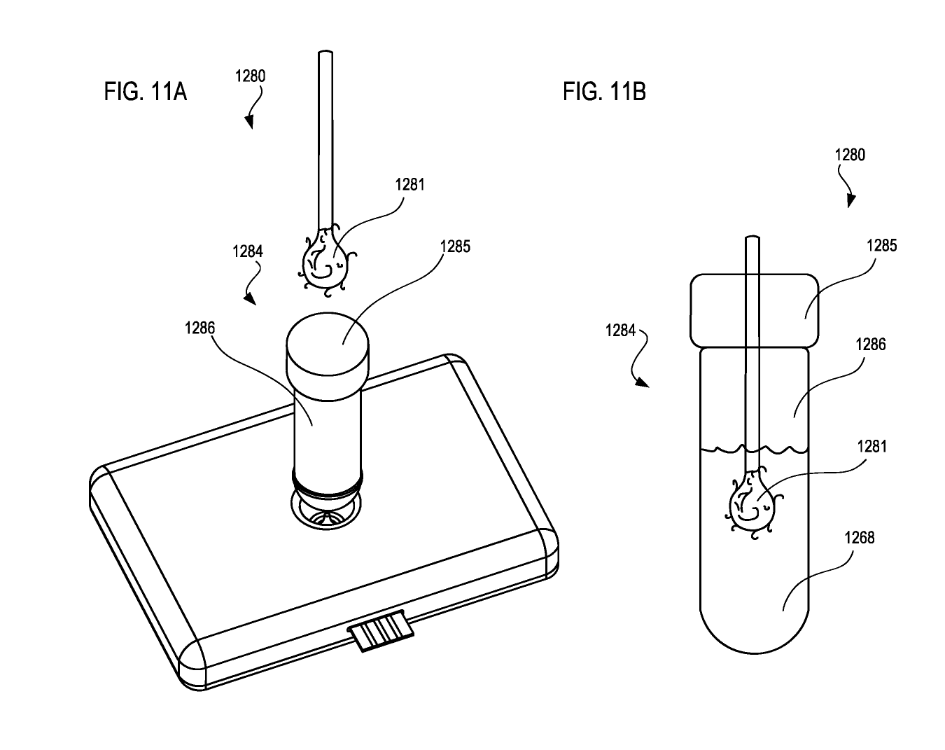

FIGS. 11A and 11B depict a sample carrier 1280, according to another embodiment of the present disclosure. As shown in FIGS. 11A and 11B, the sample carrier 1280 may comprise an absorbent swab 1281 which may be inserted into a container 1284. The container 1284 may comprise a test tube 1286 and a cap 1285. The container 1284 may also be at least partially filled with a buffer solution 1268. As illustrated in FIG. 11B, the absorbent swab 1281 may disposed within the container 1284 such that the absorbent swab 1281 is immersed in the buffer solution 1268. Immersing the absorbent swab 1281 into the buffer solution may cause the sample to elute from the absorbent swab 1281 and into the buffer solution. The buffer solution, comprising the sample, may thereafter be dispensed to the diagnostic device.

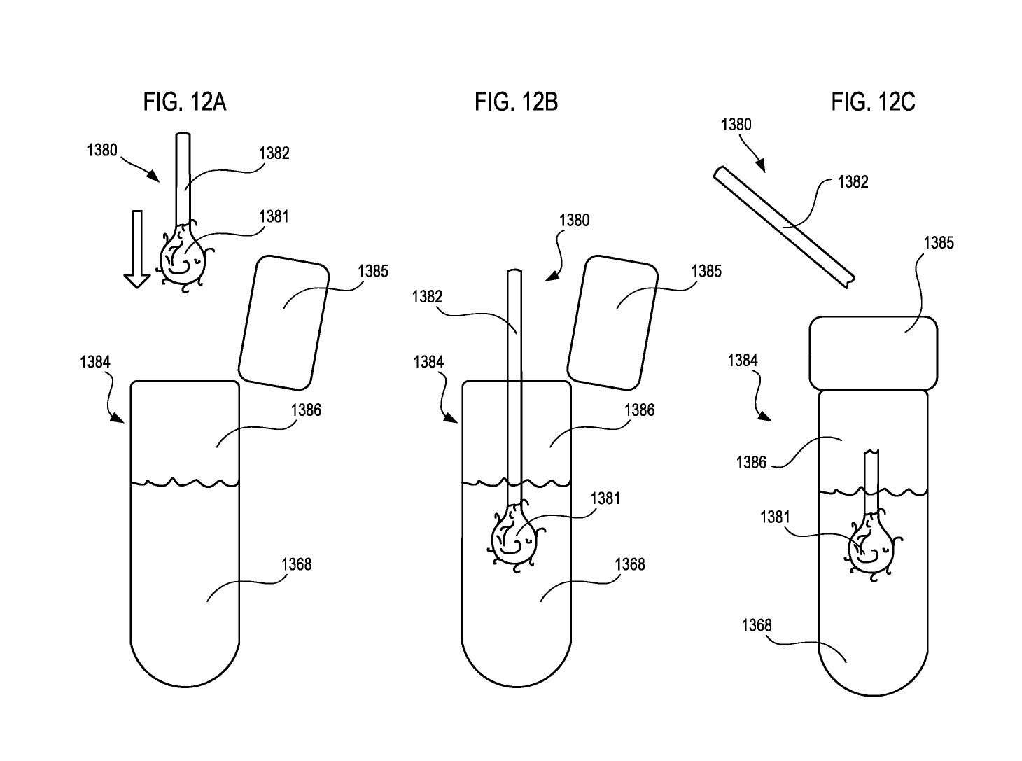

FIGS. 12A-12C depict a sample carrier 1380 according to another embodiment of the present disclosure. As shown in FIGS. 12A-12C, the sample carrier 1380 may comprise an absorbent swab 1381 and a handle 1382. The sample carrier 1380 may be inserted into a container 1384 comprising a test tube 1386 and a cap 1385. The container 1384 is also at least partially filled with a buffer solution 1368.

In FIG. 12A, the container 1384 is depicted in an open configuration in which the cap 1385 is removed and the test tube 1386 is open. While the container 1384 is in the open configuration, the sample carrier 1380 may be inserted into the test tube 1386, as indicated by the reference arrow. In FIG. 12B, the container 1384 is depicted in an open configuration and the sample carrier 1380 is partially disposed within the test tube 1386 and the buffer solution 1368. Further, a portion of the handle 1382 is shown protruding outwardly from the test tube 1386. In some embodiments, this protruding portion may be broken or otherwise removed from the sample carrier 1380 so that the cap 1385 can be used to close or seal the test tube 1386, as shown in FIG. 12C. In FIG. 12C, the container 1384 is depicted in a closed configuration in which the cap 1385 has been used to close or seal the test tube 1386. The protruding portion of the handle 1382 has been broken and removed from the sample carrier 1380, and the absorbent swab 1381 remains disposed and immersed within the buffer solution 1368 inside of the test tube 1386.

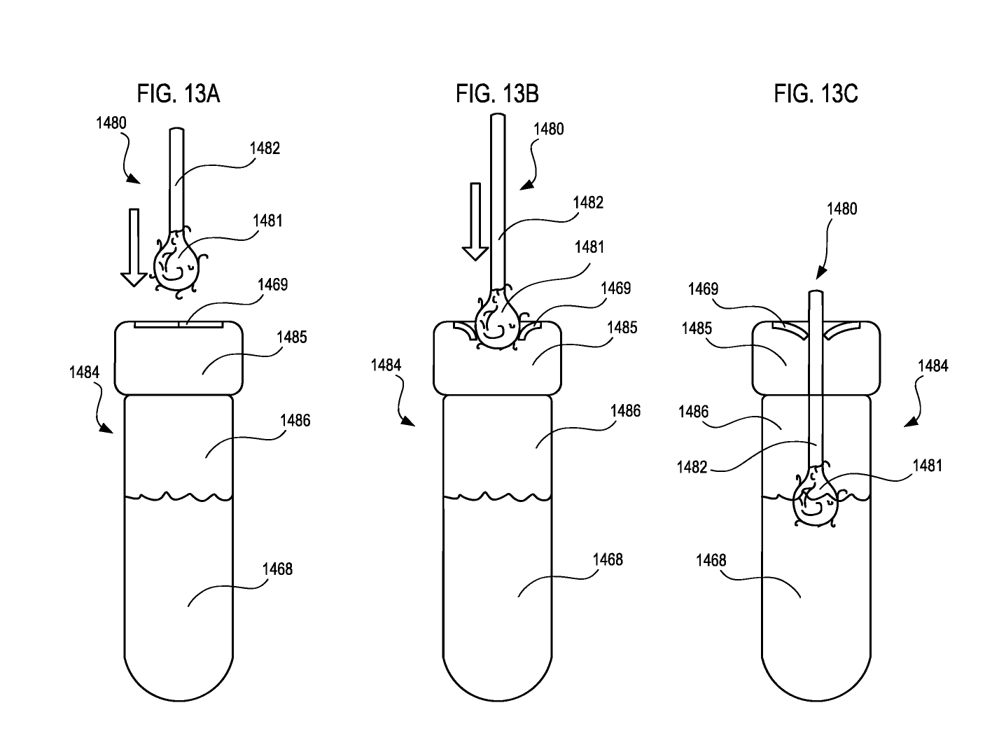

FIGS. 13A-13C depict a sample carrier 1480 according to another embodiment of the present disclosure. As shown in FIGS. 13A-13C, the sample carrier 1480 may comprise an absorbent swab 1481 and a handle 1482. The sample carrier 1480 may be inserted into a container 1484 comprising a test tube 1486 and a cap 1485. The container 1484 is also at least partially filled with a buffer solution 1468.

In the illustrate embodiment, the container 1484 further comprises a flexible membrane 1469 through which the sample carrier 1480 may be inserted. The flexible membrane 1469 may be disposed within the cap 1485 or within the test tube 1486. In FIG. 13A, the flexible membrane 1469 is closed and the sample carrier 1480 has not yet been inserted through the flexible membrane 1469. As indicated by the reference arrow, a user may insert the sample carrier 1480 inwardly into the test tube 1486 of the container 1484 and into the buffer solution 1468.

In FIG. 13B, the sample carrier 1480 has been partially inserted through the flexible membrane 1469. As indicated by the reference arrow, the sample carrier 1480 may be further inserted inwardly into the test tube 1486 of the container 1484 and into the buffer solution 1468. In FIG. 13C, the sample carrier 1480 has been inserted into the test tube 1486 and the buffer solution 1468. As further shown in FIG. 13C, the flexible membrane 1469, may close to form a seal around the handle 1482 of the sample carrier 1480 thereby preventing the buffer solution 1468 from exiting the container 1484 via the flexible membrane 1469.

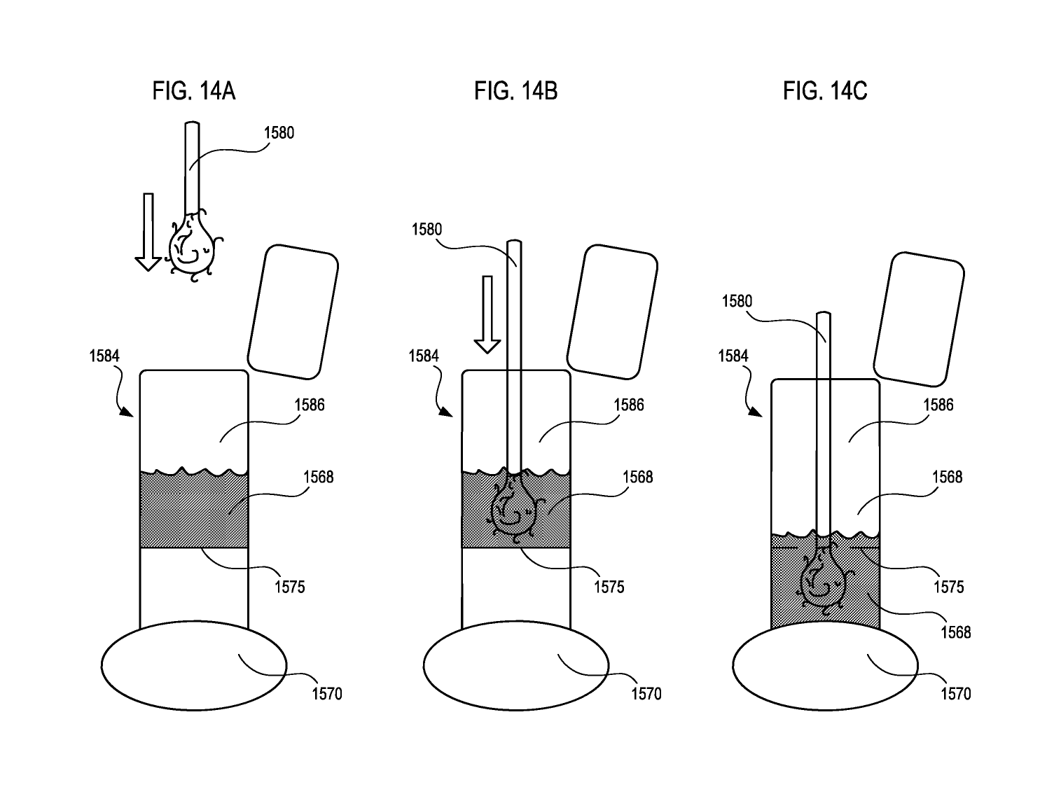

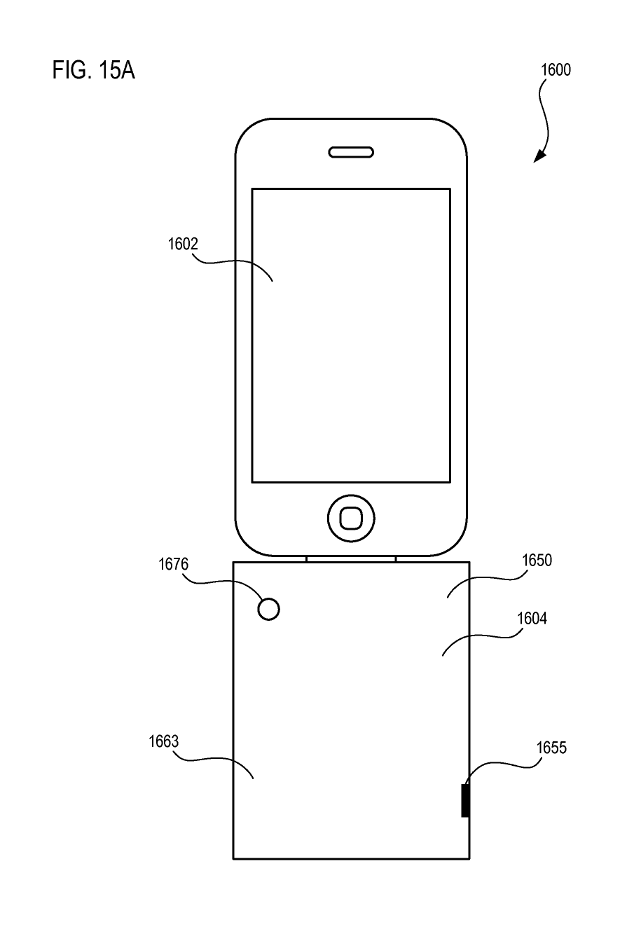

FIGS. 14A-14C depict a sample carrier 1580, according to another embodiment of the present disclosure. As shown in FIGS. 14A-14C, the sample carrier 1580 may be inserted into a container 1584 comprising a test tube 1586 that is at least partially filled with a buffer solution 1568. The container 1584 may further comprise a barrier 1575 or membrane that is configured to retain the buffer solution within a portion of the container 1584. In FIG. 14A, for example, the buffer solution 1568 is retained within a portion of the container 1584 that is away from the electrode 1570 or sensor, such that the buffer solution 1568 is prohibited from contacting the electrode 1570 or sensor until the desired time during the diagnostic test. As indicated by the reference arrow, the sample carrier 1580 may be inserted inwardly into the test tube 1586 and buffer solution 1568.

In FIG. 14B, the sample carrier 1580 has been inserted into the test tube 1586 and buffer solution 1568. The barrier 1575 remains intact and the buffer solution 1568 is still prohibited from contacting the electrode 1570 or sensor. With the sample carrier 1580 disposed or dissolved within the buffer solution, the sample may be eluted into the buffer solution. As indicated by the reference arrow, the user may further insert the sample carrier 1580 to break or rupture the barrier 1575 at the desired moment.

In FIG. 14C, the sample carrier 1580 has been forced through the barrier 1575 such that the barrier 1575 has been broken or ruptured. Once the barrier 1575 is broken, the buffer solution 1568 comprising the sample may be dispensed directly, or through one or more channels, to the electrode 1570 or sensor and a diagnostic test may be performed.

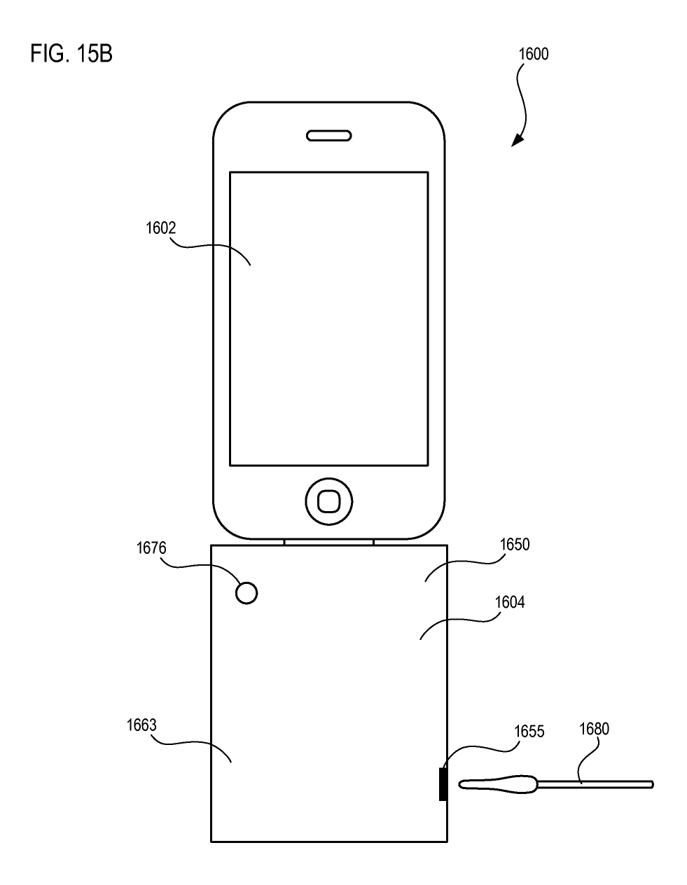

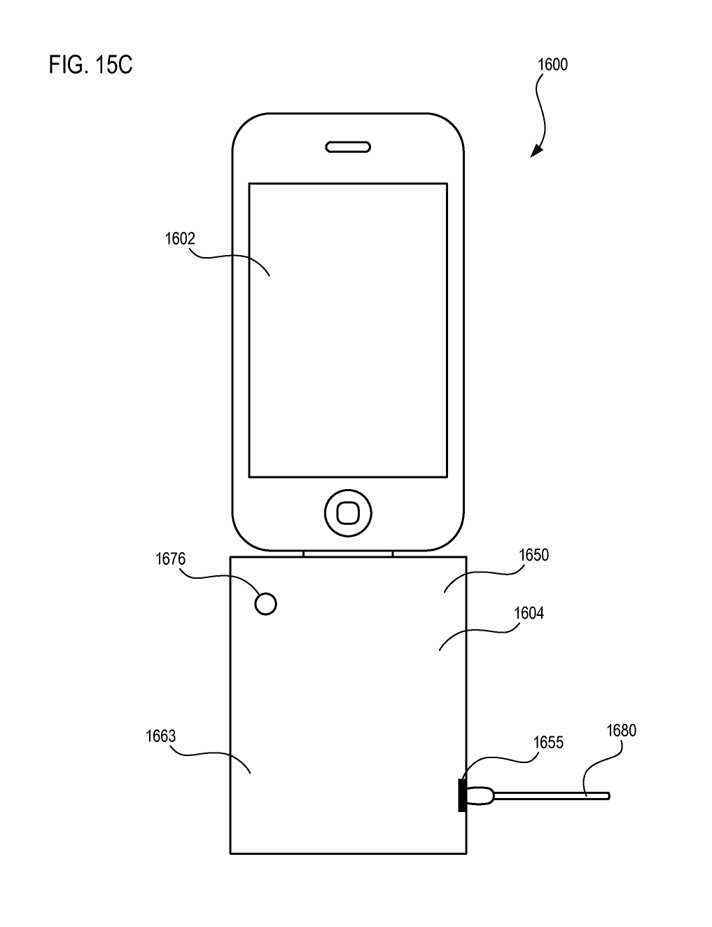

FIGS. 15A-15C depict a system 1600 for diagnostic testing, according to another embodiment of the present disclosure. As shown in the illustrated embodiment, the system 1600 comprises a PMD 1602 coupled to a diagnostic device 1604. The diagnostic device 1604 comprises a housing 1650 which includes a cover 1663. The housing 1650 further comprises a sample introduction port 1655.

In some embodiments, the housing 1655 may further comprise one or more indicators 1676. The indicator 1676 may comprise a light indicator, such as an LED indicator. The indicator 1676 may be configured to change colors and/or blink to provide the user with helpful information. For example, in some embodiments, the indicator 1676 may indicate whether the PMD 1602 is properly coupled to the diagnostic device 1604. In other embodiments, the indicator 1676 may indicate whether the diagnostic device 1604 is in a powered on or off mode. In other embodiments, the indicator 1676 may indicate the status of the diagnostic test, including whether a test is ready to begin, is currently being run, or has been completed. In other embodiments, the indicator 1676 may be configured to prompt a user to take or perform an action. In other embodiments, the indicator 1676 may indicate the results of a test (e.g., a certain color may indicate a positive test result, while a different color may indicate a negative test result).

In FIG. 15B, the system 1600 is depicted prior to performing a diagnostic test. In particular, a sample carrier 1680 is depicted prior to being inserted into the diagnostic device 1604. In FIG. 15C, the sample carrier 1680 has been inserted into the diagnostic device 1604 via the sample introduction port 1655.

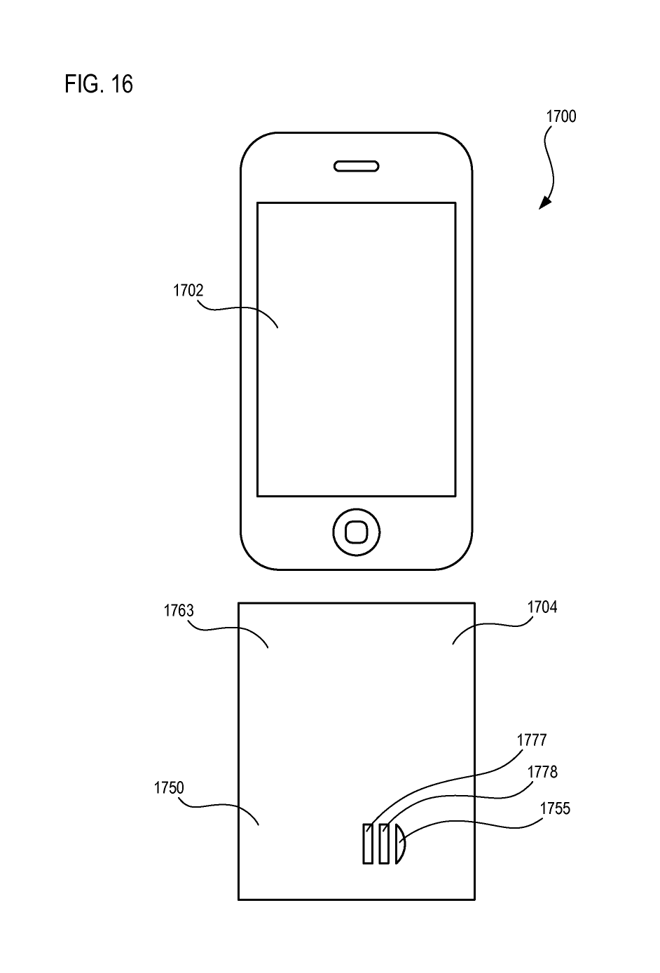

FIG. 16 depicts a system 1700 for diagnostic testing, according to another embodiment of the present disclosure. As shown in the illustrated embodiment, the diagnostic system 1700 may comprise a PMD 1702 and a diagnostic device 1704. As further shown in the illustrated embodiment, the PMD 1702 may be wirelessly coupled to the diagnostic device 1704.

The diagnostic device 1704 comprises a housing 1750 which includes a cover 1763. The diagnostic device further comprises a sample introduction port 1755 and one or more indicators 1777, 1778. As previously mentioned in relation to FIGS. 15A-15C, various types of indicators may be used.

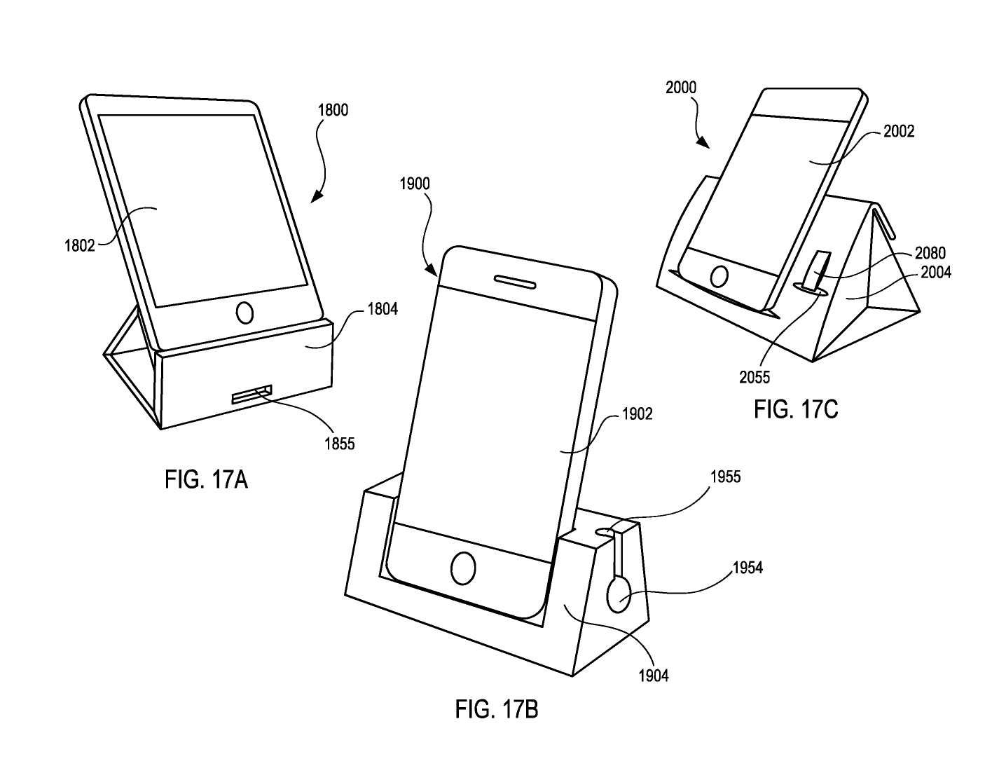

FIGS. 17A-17C depict systems 1800, 1900, 2000 for diagnostic testing, according to other embodiments of the present disclosure. As shown in FIGS. 17A-17C, in some embodiments, the diagnostic device 1804, 1904, 2004 may be configured to operate as a docking station for the PMD 1802, 1902, 2002. The docking station diagnostic device 1804, 1904, 2004 may also be configured to charge the PMD 1802, 1902, 2002.

In FIG. 17A, the diagnostic device 1804 comprises a sample introduction port 1855 which is disposed on a forward facing surface of the diagnostic device 1804. In FIG. 17B, the sample introduction port 1955 is disposed on a top wall or upper facing surface of the diagnostic device 1904. FIG. 17B further depicts a test sample chamber 1954, which may be in fluid communication with the sample introduction port 1955. The test sample chamber may be wholly, or partially, disposed within the diagnostic device 1904. In FIG. 17C, the sample introduction port 2055 is disposed on a forward facing surface of the diagnostic device 2004. The sample introduction port 2055 is also disposed such that it is adjacent to the PMD 2002. FIG. 17C further depicts a sample carrier 2080 partially disposed within the sample introduction port 2055.

A variety of systems and methods, including software implemented methods are also disclosed herein. For example, in utilizing the devices and systems disclosed herein the user may download software to the PMD. The software may provide a general operating interface for the diagnostic assay. In some embodiments, the general operating interface may be modified or supplemented by diagnostic analyte specific parameters. For example, in some embodiments, diagnostic analyte specific parameters may be derived from an external analytical sample processor and/or sensor. In other embodiments, the diagnostic analyte specific parameters may be derived from downloadable software, a barcode, or a QS tag. In yet other embodiments, the analyte specific parameters may be manually inserted.

The methods disclosed herein may be user-friendly. For example, the general and/or analyte specific diagnostic testing interface may enable a user, whether or not they are trained in laboratory assay methods, to perform a diagnostic test which may produce a test result that will be viewable on the PMD.

In some embodiment, the results of the diagnostic test may be transmitted and/or communicated to other entities. For example, the results of the diagnostic test may be transmitted to a centralized databank, a central computer in a hospital, one or more physicians or professionals who are skilled in interpreting the test results, or one or more physicians or professionals who may provide professional care in response to certain diagnostic test results. The results of the diagnostic test may also be transmitted to federal agencies such as the center for disease control (CDC) for disease epidemiological purposes, social networks, or companies that may provide products and/or services relating to the test results which may include companion pharmaceutical medications related to a particular diagnostic test. The results of the diagnostic test may also be transmitted to counselors (e.g., crisis intervention counselors for sexually transmitted diseases (STDS), pregnancy counselors, cancer counselors, etc.), geotagged resources, or other connections.

The present disclosure also relates to an interface, software environment, or other system for posting, rating, browsing, selling, purchasing, and managing of a plurality of diagnostic devices and/or the software applications that support and relate to the disclosed systems. The diagnostic devices may be designed for use through a plurality of mechanisms with the PMD on which the interface or system is housed. A user may access this system through the use of a plurality of PMDs, and may operate the diagnostic device through interaction with this interface or system.

In some embodiments, the system may be linked to an online server allowing communication between the PMD and the Internet, and may facilitate payment for and delivery of the diagnostic devices and associated software applications, transmission of data between the PMD and the server, communication for updates of content on either the online server or the PMD, and/or communication for other purposes.

The system may also provide a means by which the diagnostic devices may be controlled or manipulated by electrical signals from the PMD. The electrical signals may be caused by user-interaction with the system or may be automated as part of the system or software procured through the system.

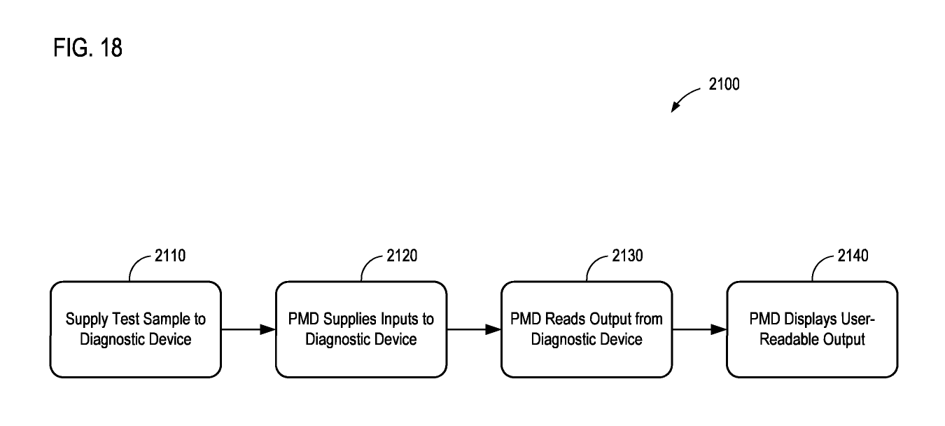

FIG. 18 is a flow chart illustrating an exemplary process 2100 of interaction between a diagnostic device and a PMD. As shown in FIG. 18, a user may supply 2110 a test sample to a sensing portion of a diagnostic device. A PMD may supply inputs 2120 to the diagnostic device, such as electrical power to run components or circuitry (i.e. pumps, sensor, transducers, etc.) and to control the function of the diagnostic device and any or all components thereof. The PMD may further read an output 2130 from the diagnostic device and display 2140 a user-readable response to the output.

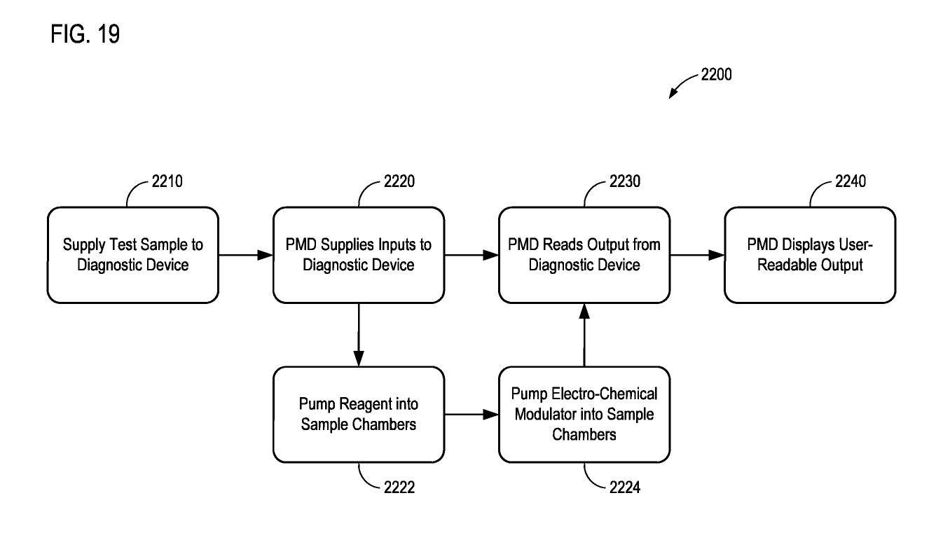

FIG. 19 is a flow chart illustrating another exemplary process 2200 of interaction between a diagnostic device and a PMD. As shown in FIG. 19, a user may supply 2210 a test sample to a sensing portion of a diagnostic device. A PMD may supply inputs 2220 to the diagnostic device, such as electrical power to run components or circuitry (i.e. pumps, sensor, transducers, etc.) and to control the function of the diagnostic device and any or all components thereof. The PMD may further read an output 2230 from the diagnostic device and display 2240 a user-readable response to the output. In some embodiments, one or more pumps may be actuated by the PMD to pump reagents 2222 and/or electro-chemical modulators 2224 into sample chambers which may be used in the diagnostic test.

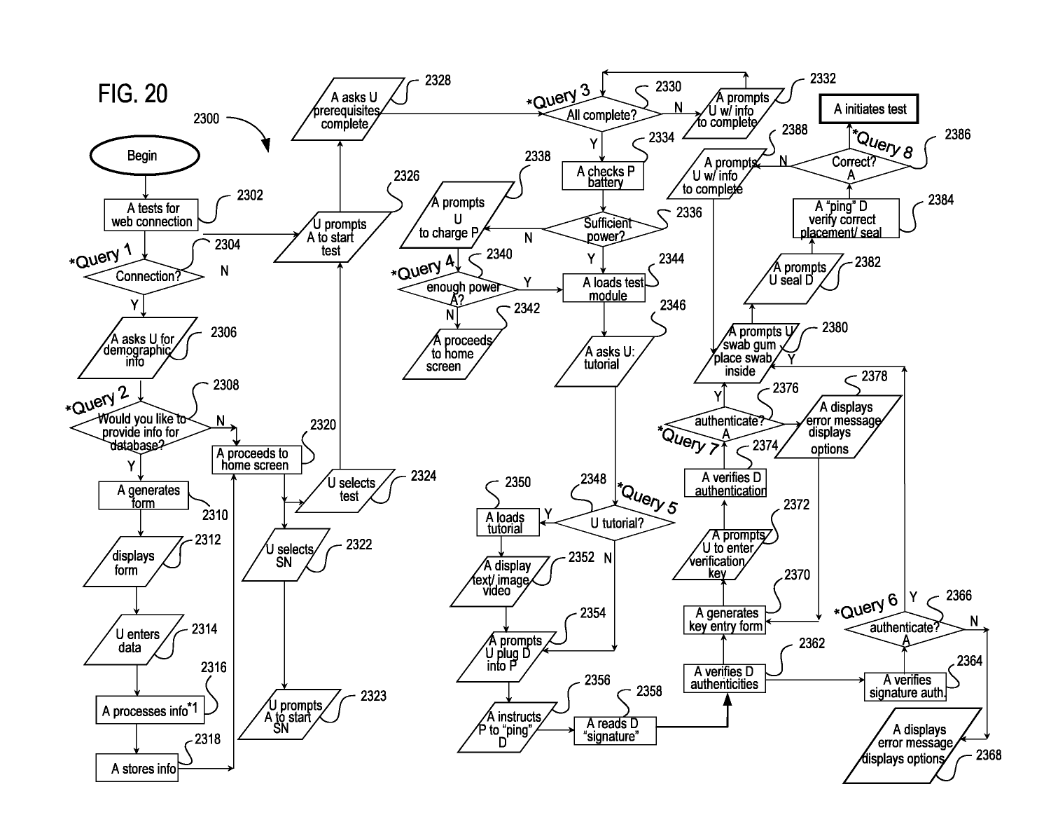

FIG. 20 depicts a flow diagram of a method 2300 for preparing for diagnostic testing, according to one embodiment of the present disclosure. The method 2300 may be performed and/or facilitated by tools, or an application comprising a plurality of tools, implemented on a PMD, or other computing device. The PMD may be coupled to an external diagnostic device, such as in the system 100 depicted in FIG. 1.

Referring to FIG. 20, a user may open a user interface of an application on a PMD. In one embodiment, the application may test 2302 for a web connection and determine 2304 (query 1) whether the PMD is connected to the internet or other network. If the PMD is connected, the application may prompt 2306 the user for demographic information. The prompt 2306 may be displayed to the user. In another embodiment, the prompt 2306 may be accomplished by an audible signal (e.g., a voice). The application may also prompt 2308 (query 2) the user to inquire whether the user would like the demographic information added to a database. If the user responds to query 2 in the affirmative, the application may generate 2310 a form and display 2312 the form to the user through the user interface. The user may then enter 2314 data into the form and submit the data and/or the form to the application, which parses or otherwise processes 2316 the data and stores 2318 it in a database to be accessed at a later time. The application may then proceed 2320 to an application home screen. If the user responds to query 2 for information in the negative, the application may proceed 2320 directly to the home screen.

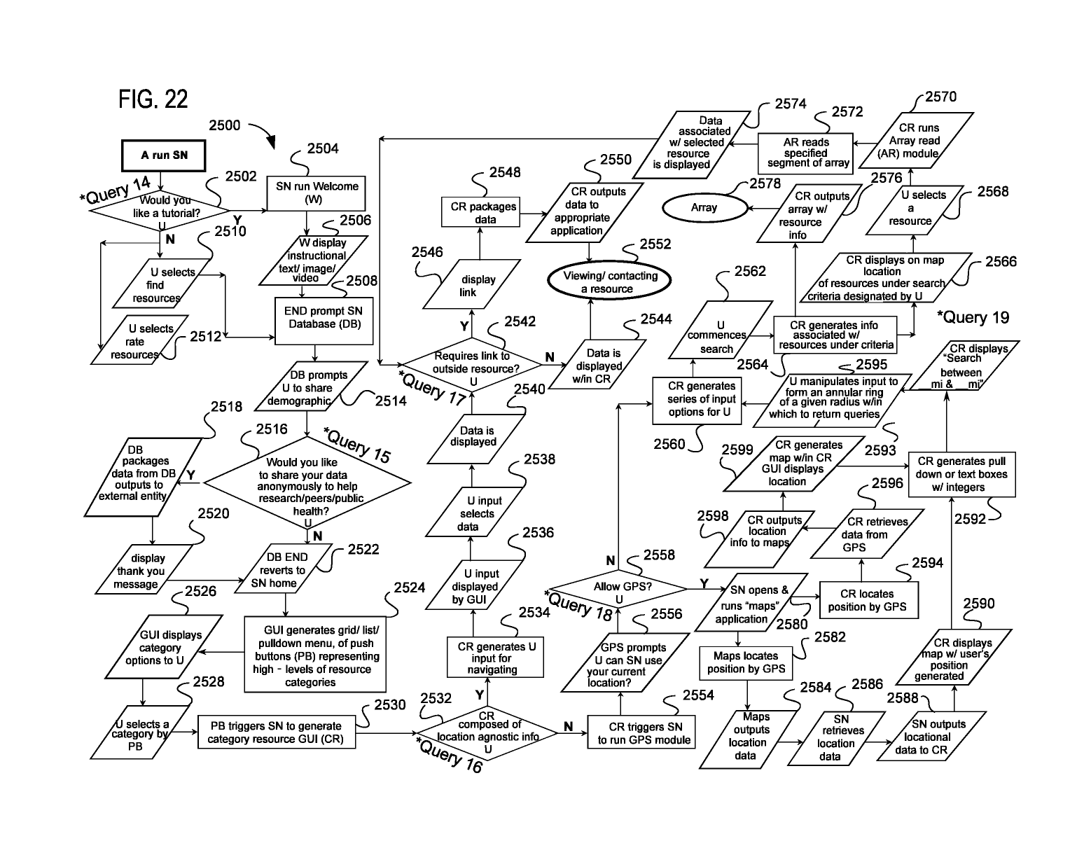

From the home screen, the user may either select 2322 to proceed to another functional area of the application, such as the “support network,” which will be discussed in greater detail below with reference to FIG. 22, or may select 2324 an option to proceed to prepare to perform a test. If the user selects 2322 to proceed to the support network, the user may prompt 2323 the application to initiate a support network module or tool. If the PMD is not connected to the internet, query 1 is answered in the negative, and the application proceeds directly to preparing to perform a test.

When the user selects the option to perform a test, the application a prompt 2326 may be provided and received to begin the test. The application may request 2328 information pertaining to one or more prerequisites to performing the test. The application may determine 2330 (query 3) whether the user has performed the necessary prerequisite tasks to perform the test. These may include questions about the last meal the user consumed, whether or not the PMD and external diagnostic device are positioned on a flat surface. If a user response in query 3 is not suitable, the application may prompt 2332 with instructions to address these responses. Questions will continue until all prerequisites are satisfied, at which time the application may proceed with the method 2300.

The application may check 2334 whether the PMD has sufficient battery power to provide the driving force for the external diagnostic device throughout the duration of the test. A determination 2336 may be made whether the PMD has sufficient power. If the PMD does not have sufficient battery power, the application may prompt 2338 the user to charge the PMD before proceeding to the test. A determination 2340 (query 4) may again be made whether the PMD has sufficient power or has been sufficiently charged. The user may elect to skip the test and proceed 2342 to the home screen (and on to the support network, if desired). If the PMD has sufficient battery power or has been charged sufficiently, the application may proceed to load 2344 a particular diagnostic test pathway or test module.

A “pre-test” module of the application may prompt 2346 the user to select whether to have a tutorial presented explaining function and procedure of the test, for example, in text, image, video, or other format. A determination 2348 (query 5) is made whether the user selected to receive the tutorial. If the user responds affirmatively to query 5, the application may load 2350 the tutorial and display 2352 and/or otherwise present the tutorial, after which the application may prompt 2354 the user to couple the external diagnostic device to the PMD, for example, by inserting the external diagnostic device into a receiving PMD port. If the user responds in the negative to query 5, the application may proceed directly to prompt 2354. The application may launch an authentication test, in which the application may “ping” 2356 the external diagnostic device, for example to read 2358 a unique authentication signature from the external diagnostic device. The application then may trigger two (possibly parallel) pathways. In one, a verification 2364 of the authentication signature is performed and a determination 2366 (query 6) may be made whether the external diagnostic device appropriately authenticates. If the external diagnostic device does not authenticate, the application may generate 2368 and display an error message, with options and instructions on how to proceed.

In the second pathway, a verification 2362 of the authentication signature is performed and the application may then generate 2370 and display a key code entry form and prompt 2372 the user to enter a verification key (found, for example, on the container in which the external diagnostic device was packaged). The verification key may be a unique code. The application may verify 2374 the device authentication. A determination 2376 (query 7) may be made whether the external diagnostic device appropriately authenticates. If this code is not accepted, the application may respond by displaying 2378 an error message, and may reset the key code entry form indefinitely, until the user enters the code that corresponds with the external diagnostic device, or returns to the home screen of the application. If this code is entered correctly and query 6 is determined in the affirmative, the application may proceed.

The application may prompt 2380 the user to utilize an absorbent swab to collect saliva from one of a few areas in the user's mouth (e.g., “Swab the gum”), The application may prompt 2382 the user to insert this swab into the external diagnostic device, and seal the swab inside the external diagnostic device. The application may “ping” the external diagnostic device to verify 2384 appropriate placement and/or positioning of the swab in the external diagnostic device and the seal by a plurality of mechanisms. A determination 2386 (query 8) is made whether the placement of the swab is correct or not. If query 8 is answered in the negative, the application may prompt 2388 the user to re-swab or to reposition the swab. If query 8 is affirmed, the application may proceed to initiate the test.

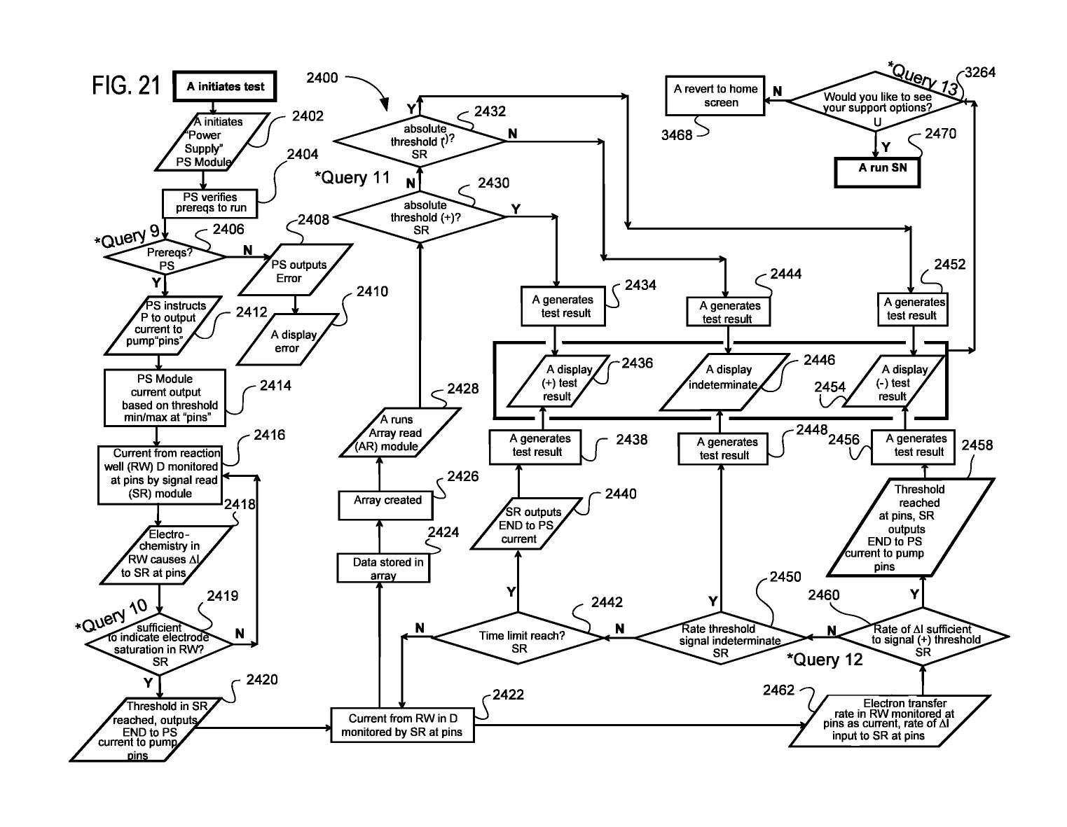

FIG. 21 depicts a flow diagram of a method 2400 for diagnostic testing, according to another embodiment of the present disclosure. At initiation of the diagnostic testing method 2400, the application may initiate 2402 a “power supply” function or controller, which may supply or otherwise control power to the external diagnostic device. This power supply function may initially perform a verification 2404 of prerequisites of the system, such as completion of the circuit through which current may be driven to power the external diagnostic device. A determination 2406 (query 9) may be made whether the prerequisites have been met. If the prerequisites have not been met, such as if the circuit is compromised, an error message may be output 2408 and/or displayed 2410 to the user.

If query 9 is affirmed, the power supply may proceed to instruct 2412 the PMD to output levels of current to pins in the PMD port that may correspond to and activate pumps in the external diagnostic device. The PMD may output 2414 the instructed current. The pumps in the external diagnostic device may introduce a fluid into reaction wells containing electrodes in the external diagnostic device, which allow a diagnostic test to occur. Current generated in the aforementioned reaction wells, which may be facilitated by the power supply function, may be monitored 2416 at the pins of the PMD port by a “signal read” function. As fluid is introduced into reaction wells, electrochemical reactions in reaction wells may cause 2418 a rate of change or electrical signal that may be monitored by the signal read function. The signal read function may consider minimum and maximum threshold levels. The minimum and maximum threshold levels may be predetermined. A determination 2419 (query 10) may be made when an electrical signal with sufficient magnitude, as measured at the pins of the PMD port corresponding to electrodes in the reaction well, may rise above the threshold level, which may indicate a saturation of the electrodes in the reaction wells. The fluid may continue to be introduced into the reaction wells until this threshold is reached, at which time the signal read function may generate an output 2420 that may trigger the power supply function to cease providing current, which may in turn stop fluid introduction.

The electrochemical reactions may continue in the reaction wells, and may be monitored 2422 by the same signal read function at the pins of the PMD port. The test may be completed by a plurality of mechanisms. In one embodiment, the electrical signal due to the concentration of a given species in the reaction well may be collected and input or stored 2424 into an array, which may be created 2426. The collection and storage 2424 may occur, possibly continuously, until an allotted time passes, at which time an “array read” function may initiate 2428 to analyze the data in this array. The values in the array may be compared against an absolute threshold of signal to determine 2430 (query 11) whether the signal constitutes a positive result and/or to determine 2432 whether the signal constitutes a negative result, or an indeterminate result. The array read function may then generate 2434, 2444, 2452, a qualitative or quantitative test result, and may package it for delivery to the application, which may then display 2436, 2446, 2454 the result to the user.

In an alternate embodiment, the signal from the reaction wells may be continuously monitored 2462, and a rate of signal change may be compared against a rate threshold as monitored by the signal read function. A given signal change rate may indicate a given concentration of some species in the reaction well, and may result in a positive determination 2460, an indeterminate determination 2450, or a negative determination 2442. Each threshold may trigger 2440, 2458 the power supply function to cease, and the signal read function may generate 2438, 2448, 2456 and/or package the result for delivery to the application, which may then display 2436, 2446, 2454 the result to the user.

A prompt may be presented 2464 (query 13) to the user to decide whether to proceed 2470 to the support network, or to return 2468 to the application's home screen. The user may select the option to proceed 2470 to the support network, which may trigger the application to initiate the support network function.

FIG. 22 depicts a flow diagram of a method 2500 of accessing a support network, based on diagnostic testing, according to another embodiment of the present disclosure. In FIG. 22, the support network may commence by generating and/or displaying a prompt 2502 (query 14) asking the user if they would like to see a tutorial about the support network. If the user responds to query 14 in the affirmative, the support network may run 2504 a “welcome” function, which may generate and display 2506 text, images, video, or other material, which may provide the user information. The text, images, video, or other material may be displayed 2506 on the PMD, for example. A “database” function may be triggered 2508, which may refer to demographic information, information aggregated during the test, or other information.

If the user responds to query 14 in the negative, the user may be presented with several options, including a presentation 2510 of an option to proceed directly to finding resources, in which the support network may also trigger 2508 the database function. The user may also decide to proceed 2512 directly to rating resources that the user may have previously utilized or otherwise interacted with. A listing of previously utilized resources, may be collected, for example, in a folder separate from the rest of the support network, or may be accessed by another mechanism.

Once triggered 2508, the database function may generate and display a prompt 2514 to the user, which may ask 2516 (query 15) whether the user would like to share data gathered throughout the test anonymously or non-anonymously that may benefit public health institutions, research organizations, peer support groups, or other groups. If the user responds in the affirmative, the database may access the various forms submitted or arrays populated by the user or the application, and may package 2518 these data, and generate an output to be transmitted to external entities by a plurality of mechanisms. The support network may then generate and display 2520 a message thanking the user.

The database function may subsequently terminate 2522, which may trigger the generation 2524 and/or presentation of a graphical user interface (GUI). If the user responds to query 15 in the negative, the method 2500 may proceed directly to generation 2524 and/or presentation of this GUI, which may generate a grid, list, pulldown menu, pushbuttons, or other user input options. These options may be displayed 2526 and may represent a high-level variety of resource categories. A user input selection 2528 may be received that indicates a user selection of one of the resource categories, which I may trigger 2530 the generation and display (in text, image, video, or other format), for example, of another GUI or window containing specific resources within a selected resource category. A variety of input mechanisms may also be present in this GUI or window.

The support network may determine 2532 whether these displayed category resources are composed of location-agnostic information or resources. If these resources are location-agnostic, the category resource may generate 2534 a specific set of user input mechanisms by which the user may select specific resources. The user input mechanisms may be displayed 2536, for example on a GUI. User input received, for example through the GUI, may select 2538 a resource, which may be displayed 2540, and the category resource may determine 2542 (query 17) whether the selected resource is housed within the category resource, or if a link to an outside resource may be required. If query 17 is negative, the category resource may retrieve and display 2544 the data associated with the specific resource within the support network environment. If query 17 is determined to be affirmative, the category resource may generate and display 2546 to the user a link, contact information, or other mechanism that may direct the user out of the support network. Direction outside of the support network may also trigger a “referral” function which may trace the user's contact, and store record of the referral. Data may be packaged 2548 and output 2550 to the resource and/or an appropriate application. The resource information may be presented for viewing 2552 and/or contacting.

If the response to query 16 is that resources may be location-specific, the category resource may trigger 2554 the support network to run a “GPS” function, which may generate and display a prompt 2556 to the user asking whether the support network may use the user's current location. A determination 2558 (query 18) is made whether GPS is allowed. If query 18 is affirmed, one of two pathways may be taken. In one embodiment, the support network may be triggered to open 2580 and/or run a “Maps” application, which may locate 2582 the user's location through a GPS hardware, software, or system located, for example, in the PMD. This GPS may then, through the “Maps” application, output 2584 data about the user's location, which may be retrieved 2586 by the support network and may be outputted 2588 to the category resource, triggering a map generation and display 2590. This display 2590 of a map may allow the user to visualize their position in relation to resources around them. The category resource may also generate 2592 input mechanisms and prompt 2593 (query 19) the user to define a search area.

In an alternate embodiment, after the user has responded to query 18 in the affirmative, the support network may open 2580 and run a “Maps” application. The category resource may directly utilize the GPS system of the PMD to locate 2594 the user's position and retrieve 496 this datum. The category resource may then output 2598 this location information to a map generated 2599 within the category resource, and may also display the map to allow the user to visualize their geographic location. The category resource may also generate 2592 input mechanisms and prompt 2593 (query 19) the user to define a search area.

The category resource may generate and display, with query 19, user input options that allow the user to manipulate 2595 the input options to specify an annular area of a given inner and outer radius (i.e. between 5 and 25 miles from the user) from which to return resource results. This path may then converge with the pathway from a negative answer to query 18, and the category resource may generate 2560 and display other options for specifying the criteria of resources returned and displayed to the user. The user may then commence a search 2562, triggering the category resource to compare all resources in the category against the user's specifications. The category resource may then retrieve and/or generate 2564 information associated with those resources that align with the user's criteria, and may display 2566 them on a map, with or without a reference point showing the user's position, and therefore provide either geo-specific or location-agnostic resources. In the process of retrieving data about the resources, the category resource may also populate or otherwise output 2576 an array 2578 with the information gathered from the search. When the user selects 2568 a given resource, an “array read” function may be triggered 2570, which may read 2572 data in the generated array, aggregate the data, package it, and output it to the category resource. The category resource may then display 2574 the data. This pathway may then converge on query 17, and may follow similar pathways.

### EXAMPLES

### Example 1

A diagnostic testing surface was prepared by immobilizing a peptide comprising methylene blue, an antibody capture probe specific to HIV-1 capsid protein, p24, a hydrocarbon chain, and a thiol group onto the surface of an electrode of a diagnostic device according to the present disclosure. A sample solution was prepared by eluting an HIV-1 capsid protein, p24 in a physiological buffer solution containing Tris, NaCl, KCl, MgCl₂, and CaCl₂ which mimics the chemical environment of blood serum. A control solution was also prepared consisting of the physiological buffer solution in the absence of the HIV-1 capsid protein, p24.

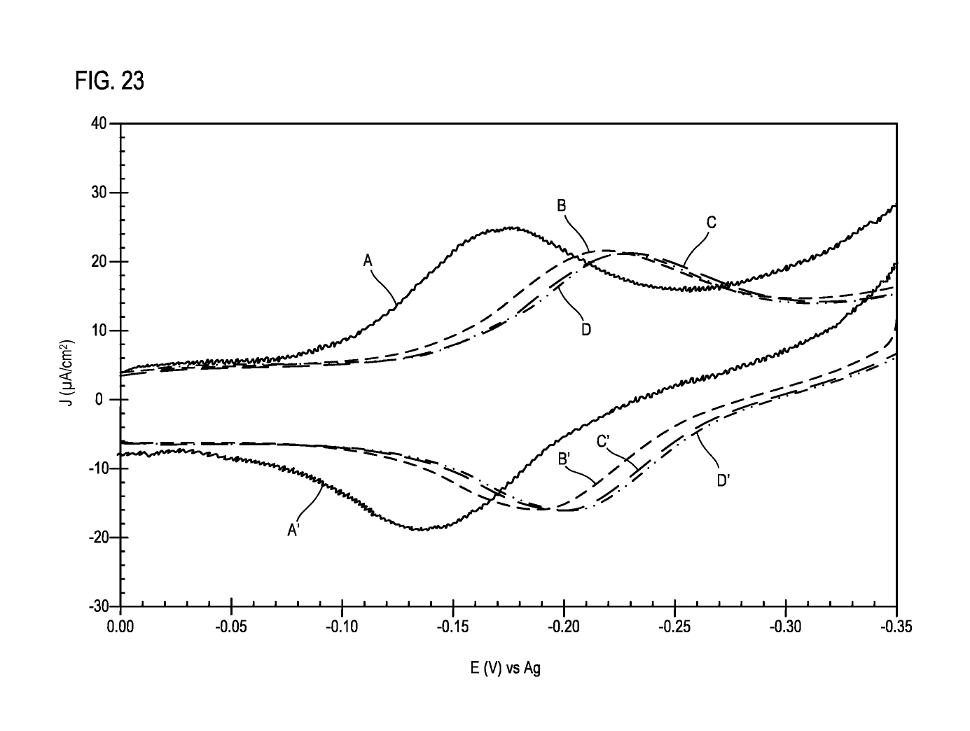

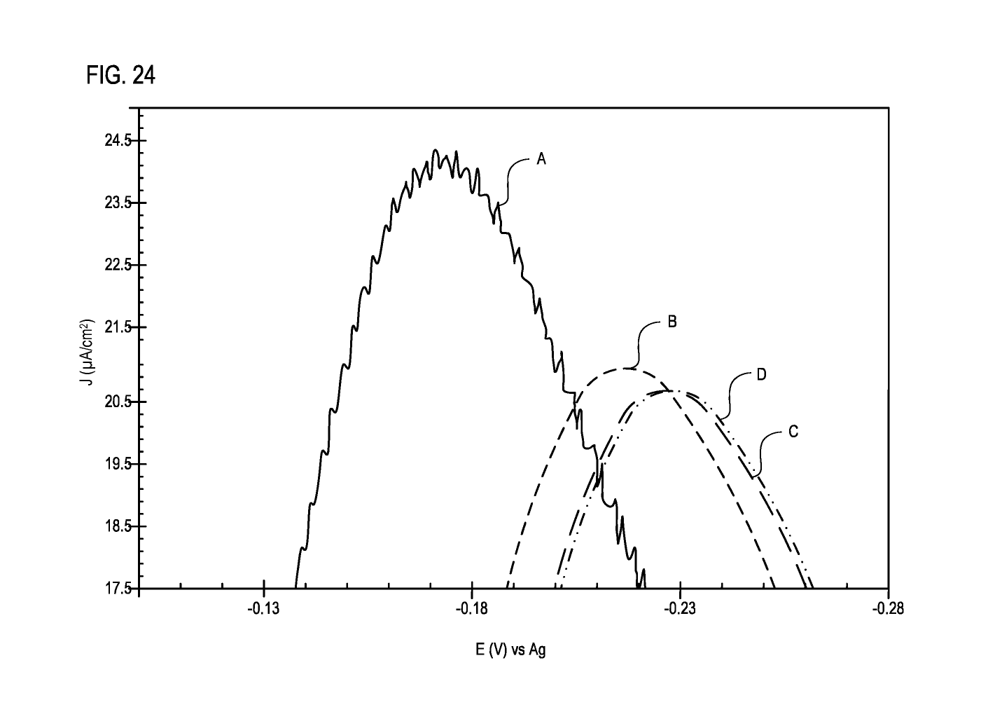

An aliquot of the sample solution was dispensed onto the diagnostic testing surface and the current density was measured using cyclic voltammetry, a procedure wherein the current density is measured as the voltage input is varied over time. An aliquot of the control solution was separately dispensed onto a diagnostic testing surface and the current density was also measured using cyclic voltammetry. The measured current densities of the sample solution and the control solution are illustrated in the graph of FIGS. 23 and 24, where FIG. 24 is an enlarged portion of the graph of FIG. 23.

In FIGS. 23 and 24, line A represents the current density of the control sample, and lines B, C, and D represent the current density of the sample solution taken after 5, 10, and 20 minutes, respectively. Lines A, B, C, and D represent measurements of the current densities that were taken during a cycle where the voltage was being lowered. Lines A′, B′, C′, and D′ represent measurements of the current densities that were taken during a cycle where the voltage was being raised.

The difference between the current densities (i.e., the height of the peaks) of the control solution and the sample solution represents the change in current caused by a conformational change on the diagnostic testing surface. The current densities (i.e., electrical signals) may be transmitted from the diagnostic device and to a PMD for processing into a qualitative diagnostic result, a quantitative diagnostic test result, or both.

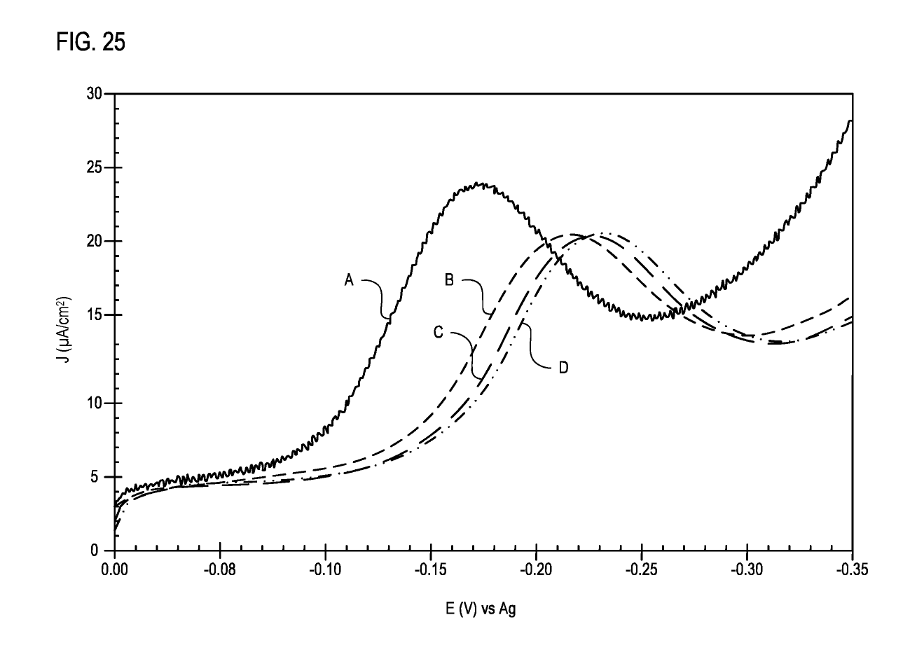

A second aliquot of the sample solution was dispensed onto the diagnostic testing surface and the current density was measured using cyclic voltammetry. A second aliquot of the control solution was also separately dispensed onto a diagnostic testing surface and the current density was also measured using cyclic voltammetry. The measured current densities of the sample solution and the control solution are illustrated in the graph of FIG. 25.

### Example 2

A diagnostic testing surface was prepared by immobilizing a peptide comprising an antibody capture probe specific to PBP2a protein onto the surface of an electrode of a diagnostic device according to the present disclosure. A sample solution was prepared by eluting a PBP2a protein in a physiological buffer solution containing Tris, NaCl, KCl, MgCl₂, and CaCl₂ which mimics the chemical environment of blood serum. A control solution was also prepared consisting of the physiological buffer solution in the absence of the PBP2a protein.

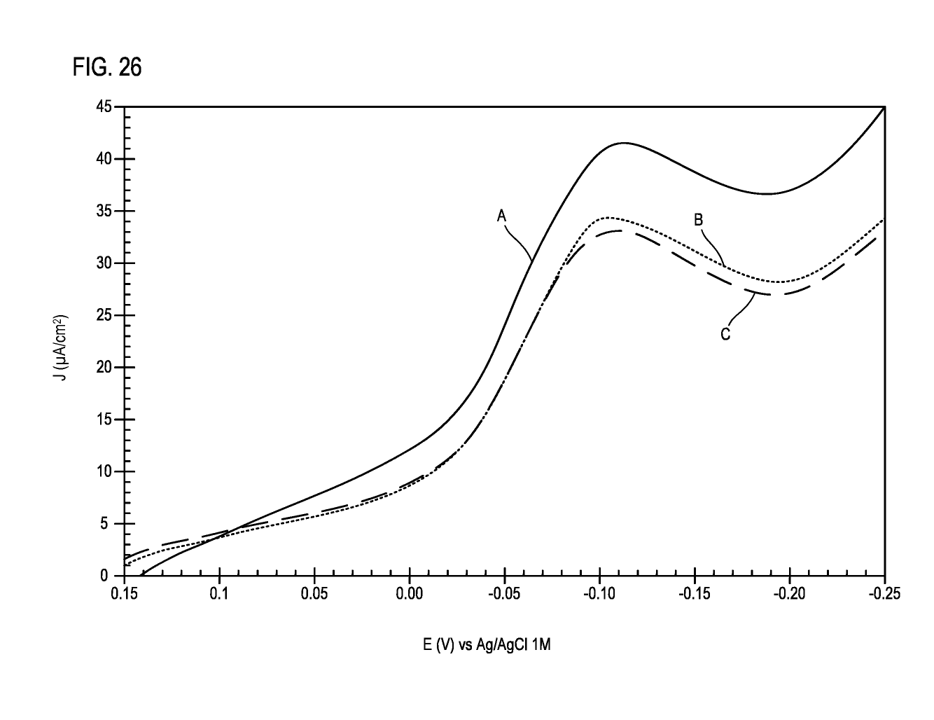

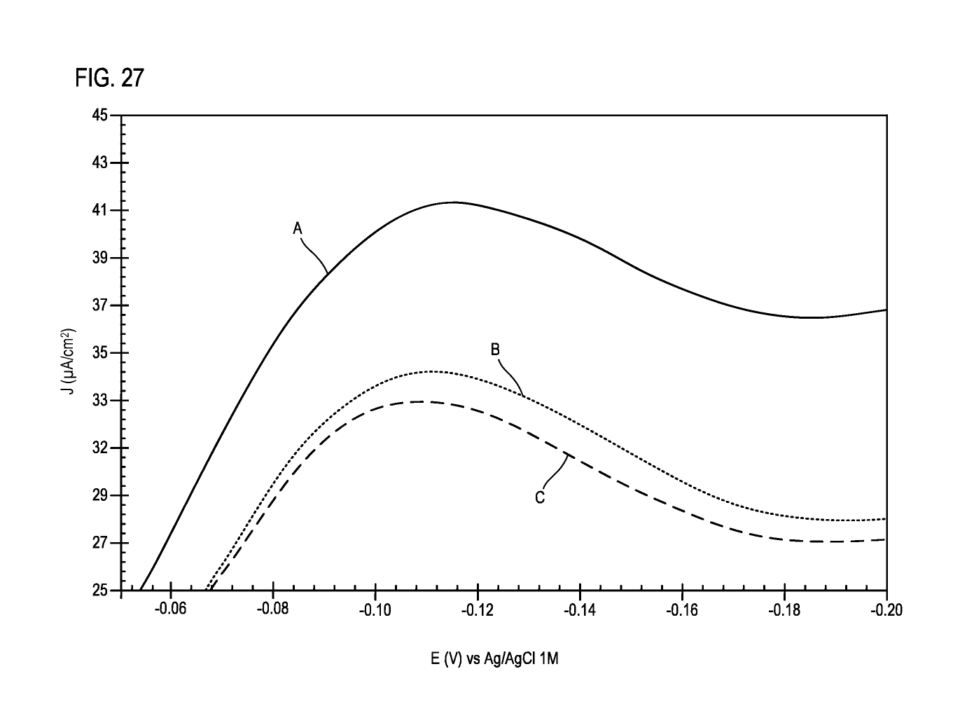

An aliquot of the sample solution was dispensed onto the diagnostic testing surface and the current density was measured using cyclic voltammetry, a procedure wherein the current density is measured as the voltage input is varied over time. An aliquot of the control solution was separately dispensed onto a diagnostic testing surface and the current density was also measured using cyclic voltammetry. The measured current densities of the sample solution and the control solution are illustrated in the graph of FIGS. 26 and 27, where FIG. 27 is an enlarged portion of the graph of FIG. 26.

In FIGS. 26 and 27, line A represents the current density of the control sample, and lines B and C represent the current density of the sample solution taken after 5 and 10 minutes, respectively. The difference between the current densities (i.e., the height of the peaks) of the control solution and the sample solution represents the change in current caused by a conformational change on the diagnostic testing surface. The current densities (i.e., electrical signals) may be transmitted from the diagnostic device and to a PMD for processing into a qualitative diagnostic result, a quantitative diagnostic test result, or both.

The present disclosure has been made with reference to various exemplary embodiments including the best mode. However, those skilled in the art will recognize that changes and modifications may be made to the exemplary embodiments without departing from the scope of the present disclosure. For example, various operational steps, as well as components for carrying out operational steps, may be implemented in alternate ways depending upon the particular application or in consideration of any number of cost functions associated with the operation of the system, e.g., one or more of the steps may be deleted, modified, or combined with other steps.

Additionally, as will be appreciated by one of ordinary skill in the art, principles of the present disclosure may be reflected in a computer program product on a tangible computer-readable storage medium having computer-readable program code means embodied in the storage medium. Any suitable computer-readable storage medium may be utilized, including magnetic storage devices (hard disks, floppy disks, and the like), optical storage devices (CD-ROMs, DVDs, Blu-Ray discs, and the like), flash memory, and/or the like. These computer program instructions may be loaded onto a general purpose computer, special purpose computer, or other programmable data processing apparatus to produce a machine, such that the instructions that execute on the computer or other programmable data processing apparatus create means for implementing the functions specified. These computer program instructions may also be stored in a computer-readable memory that can direct a computer or other programmable data processing apparatus to function in a particular manner, such that the instructions stored in the computer-readable memory produce an article of manufacture including instruction means which implement the function specified. The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions specified.

While the principles of this disclosure have been shown in various embodiments, many modifications of structure, arrangements, proportions, the elements, materials and components, used in practice, which are particularly adapted for a specific environment and operating requirements may be used without departing from the principles and scope of this disclosure. These and other changes or modifications are intended to be included within the scope of the present disclosure.

The foregoing specification has been described with reference to various embodiments. However, one of ordinary skill in the art appreciates that various modifications and changes can be made without departing from the scope of the present disclosure. Accordingly, this disclosure is to be regarded in an illustrative rather than a restrictive sense, and all such modifications are intended to be included within the scope thereof. Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, required, or essential feature or element. As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. Also, as used herein, the terms “coupled,” “coupling,” or any other variation thereof, are intended to cover a physical connection, an electrical connection, a magnetic connection, an optical connection, a communicative connection, a functional connection, and/or any other connection.

It will be obvious to those having skill in the art that many changes may be made to the details of the above-described embodiments without departing from the underlying principles of the disclosure.

1. A system for diagnostic testing, comprising:
a portable multifunctional device configured to transmit and receive electrical signals; and
a diagnostic device coupled to the portable multifunctional device, the diagnostic device being configured to perform a diagnostic test on a test sample and generate an electrical signal in response to the diagnostic test, the diagnostic device comprising:
a housing;
a sample introduction port configured to receive the test sample; and
a test sample chamber configured to retain the test sample during the diagnostic test.

2-14. (canceled)

15. The system of claim 1, wherein the sample introduction port is in fluid communication with the sample chamber via one or more channels such that the test sample may be inserted through the sample introduction port and into the sample chamber.

16. The system of claim 1, wherein the test sample is retained within a sample carrier.

17. The system of claim 16, wherein the sample carrier comprises an absorbents swab.

18. The system of claim 16, wherein the sample carrier comprises a capillary tube.

19. The system of claim 16, wherein the sample carrier comprises a terminating loop and a handle.

20. The system of claim 16, wherein the sample carrier may be disposed within a container containing a buffer solution that is configured to elute the sample from the sample carrier.

21. The system of claim 1, wherein the diagnostic device further comprises a reagent chamber that is in fluid communication with the test sample chamber via one or more channels.

22. The system of claim 21, wherein the reagent chamber is configured to retain a buffer solution.

23. The system of claim 1, wherein the diagnostic device further comprises an electronically-activated polymer that is configured to expand and contract in response to an electrical stimuli.

24. The system of claim 23, wherein the electronically-activated polymer comprises an electroactive polymer.

25. The system of claim 23, wherein the electronically-activated polymer comprises an ionic polymer metal composite.

26. The system of claim 23, wherein the electronically-activated polymer is configured to move materials within the diagnostic device.

27. The system of claim 23, wherein the electronically-activated polymer is configured to mix materials within the diagnostic device.

28. The system of claim 1, wherein the diagnostic device further comprises a user-operated pump that is configured to expand and contract in response to a manual stimulation.

29. The system of claim 28, wherein the user-operated pump is configured to move materials within the diagnostic device.

30. The system of claim 28, wherein the user-operated pump is configured to mix materials within the diagnostic device.

31. (canceled)

32. The system of claim 31, wherein the indicator is an LED indicator.

33. The system of claim 31, wherein the indicator is configured to inform a user of the status of a diagnostic test.

34. A system for diagnostic testing, comprising:
a computing device selected from a desktop computer or a portable computer, wherein the computing device is configured to transmit and receive electrical signals; and
a diagnostic device coupled to the computing device, the diagnostic device being configured to perform a diagnostic test on a test sample and generate an electrical signal in response to the diagnostic test, the diagnostic device comprising:
a housing;
a sample introduction port configured to receive the test sample; and
a test sample chamber configured to retain the test sample during the diagnostic test.
